(19) United States
(12) Patent Application Publication
(10) Pub. No.: US 2022/0251071 A1
(43) Pub. Date: Aug. 11, 2022

(54) METHOD TO MAKE SMALL-MOLECULE MURINE DOUBLE MINUTE 2 PROTEIN (MDM2)-DEGRADING COMPOUNDS, COMPOUNDS FORMED THEREBY, AND PHARMACEUTICAL COMPOSITIONS CONTAINING THEM

(71) Applicant: Wisconsin Alumni Research Foundation, Madison, WI (US)

(72) Inventors: Weiping Tang, Middleton, WI (US); Bo Wang, Middleton, WI (US); Suzhen Wu, Madison, WI (US); Ka Yang, Madison, WI (US)

(21) Appl. No.: 17/610,896

(22) PCT Filed: May 13, 2020
(86) PCT No.: PCT/US2020/032664

§ 371 Date: Nov. 12, 2021

Related U.S. Application Data
(60) Provisional application No. 62/847,357, filed on May 14, 2019.

(51) Int. Cl. C07D 401/14; A61K 31/496; A61P 35/00

(52) U.S. Cl. CPC C07D 401/14; A61P 35/00; A61K 31/496

(57) ABSTRACT

Compounds of the formula:

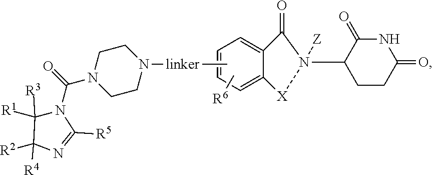

pharmaceutical compositions containing the compounds, and methods of inhibiting neoplastic cell growth using the compounds and compositions.

### CROSS-REFERENCE TO RELATED APPLICATIONS

Priority is hereby claimed to U.S. provisional patent application Ser. No. 62/847,357, filed May 14, 2019, which is incorporated herein by reference.

### FEDERAL FUNDING STATEMENT

This invention was made with government support under GM120357 awarded by the National Institutes of Health. The government has certain rights in the invention.

### BACKGROUND

Tumor suppressor protein p53, plays a pivotal role in the regulation of cell processes and the prevention of cancer development[1, 2]. It activates key regulators that control the cell cycle, DNA repair and programmed cell death. However, the loss of function of p53 can result from mutations or deletions[3], which occur in approximately half of the human cancers.

Murine double minute 2 (MDM2) is a negative endogenous regulator of p53, and its regulatory activity proceeds through three primarily pathways[4, 5]: 1) MDM2 binds the transactivation domain of p53, which prohibits p53 from binding to its targeted gene, resulting in p53's inability to function as a transcription factor; 2) MDM2 ubiquitinates p53 upon binding, which promotes p53 degradation in the proteasome; 3) MDM2 exports p53 out of the cell nucleus to prevent the transcriptional activity of p53. In some cancerous cells, MDM2 is overexpressed or the MDM2 gene is amplified. To restore the tumor suppressor function of p53, disruption of the MDM2-p53 interactions has become a promising therapeutic strategy for the p53 wild-type human cancers.

The interactions between MDM2 and p53 were unveiled unambiguously by the high-resolution co-crystal structure, depicted in FIG. 1A[6]. p53 has an alpha-helical conformation that binds to MDM2 within MDM2's hydrophobic clef. MDM2 primarily interacts with p53 through three amino acid residues: Leu26, Trp23, and Phe19. By mimicking the interactions between MDM2 and p53, dozens of small molecule inhibitors for MDM2 have been developed[7-19]. Among them, the co-crystal structures of several inhibitors with MDM2 have been solved [20-25]. Four representative clinical candidates are shown in FIG. 1B and moieties that mimic Leu26, Trp23, and Phe19 are also indicated.

Despite the significant progress on the development of MDM2 inhibitors, small molecule MDM2 inhibitors have significant limitations, including drug resistance and accumulation issues [26]. Wang and co-workers have shown that a single dose of MDM2 inhibitor (SAR405838) only induced a few hours of p53 accumulation in xenograft tumor tissues, suggesting that p53 may be resistant to MDM2 inhibitors[25]. In addition, inhibition of p53 may lead to overexpression and accumulation of MDM2 in normal tissues, which may lead to toxicity issues. With these limitations in mind, researchers have looked toward other methods to modulate MDM2 levels.

As an alternative to small molecule inhibitors, a novel therapeutic approach has been developed, termed proteolysis-targeting chimeras (PROTACs). PROTACs were formally introduced as a therapeutic option in 2001 [27], and they take advantage of the ubiquitin-proteasome system (UPS) in mammalian cells. PROTACs (also known as degraders) are heterobifunctional molecules with two ligands connected by an appropriate linker (FIG. 2A). One ligand binds to a protein of interest/target protein, and the other ligand binds to an E3 ubiquitin ligase. This bifunctional molecule then positions the E3 ligase in close proximity to the target protein to form a ternary complex. This complex subsequently promotes polyubiquitination of the target protein, thereby marking the target protein for degradation in the proteasome. Since the discovery of small molecule ligands for E3 ubiquitin ligases, including Von Hippel Lindau (VHL)[28-30], cereblon (CRBN)[31-33], and inhibitor of apoptosis protein (IAP)[34-36], this technology has demonstrated tremendous success for the degradation of a number of disease-causing proteins, such as BCR-ABL[34, 37], BET and BRD[31, 38-43], estrogen receptors[44], HDAC6[45] and cyclin-dependent kinases[46-48], amongst others.

Recently, the Wang group reported the first MDM2 degrader by tethering their own spirooxindole MDM2 inhibitor, MI-1061, to lenalidomide[49]. This MDM2 degrader promoted effective degradation of MDM2 and exhibited promising anti-tumor effects. MDM2 inhibition was first achieved by using readily-available nutlin derivatives[50]. By conjugating nutlin derivative 1 and lenalidomide analogue 2 with linkers with variable lengths, we have prepared a series of novel MDM2 degraders (FIG. 2C).

In RS4;11 leukemia cells that feature wild-type p53, low nanomolar concentrations of PROTAC 32 induced efficient degradation of MDM2 and stabilization of p53. Furthermore, 32 significantly inhibited the proliferation and induced apoptosis of RS4;11 cells. In terms of anti-proliferation effects, degrader 32 is almost 1,000-fold more potent than the corresponding MDM2 inhibitor. We believe the potent activity of 32 can be attributed to its short linker length. Degrader 32 has only one acetylene and one methylene unit between the benzene ring of lenalidomide and the ligand for the target protein, which represents one of the shortest linkers discovered to date for PROTACs[32, 46, 49, 51].

### SUMMARY

Tumor suppressor protein p53, is important to the regulation of cell processes and the prevention of cancer development. In some cancer cells, the function of p53 is inhibited by murine double minute 2 protein (MDM2). To restore the function of p53, the inhibition or depletion of MDM2 has been become a potential therapeutic treatment. We have successfully developed a series of small molecule MDM2 degraders that can promote the proteolysis of MDM2 oncoprotein, thus reactivating tumor suppressor p53. The superior degrader features a MDM2 ligand and a cereblon E3 ubiquitin ligase ligand with a linker between the ligands. At low nanomolar concentrations in RS4;11 leukemia cells, these degraders promote efficient degradation of MDM2. They also inhibit the proliferation of leukemia cells with an IC₅₀ value of about 3.2 nM or less and induces apoptosis effectively. All of these data indicate that the MDM2 degraders disclosed herein are useful therapeutics for inhibiting the growth of neoplastic cells, including cancer cells such as leukemia cells.

Disclosed herein are compounds selected from the group consisting of:

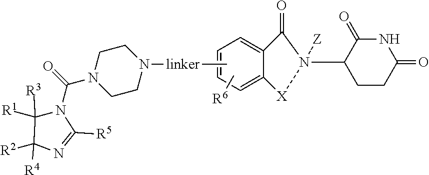

wherein each R¹, R², R³, R⁴, R⁵, and R⁶ are independently hydrogen or a substituent selected from the group consisting of: hydrogen, alkyl, alkenyl, alkynyl, alkoxy, halo, haloalkyl, hydroxy, hydroxyalkyl, aryl, alkyl-substituted aryl, alkoxy-substituted aryl, halo-substituted aryl, aroyl, (aryl)alkyl, heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl(alkyl)amine, and cyano;

the dashed lines attached to X and Z are single bonds or are absent; when the dashed line attached to X is a single bond, X is —(C═O)— or —CH₂— and Z and the dashed line attached to Z are absent; when the dashed line attached to X is absent, X is hydrogen, Z is a hydrogen, and the dashed line attached to Z is a single bond;

“linker” is selected from the group consisting of a single bond, a C₁-C₁₂-alkylene, akenylene, or akynylene, —(C═O)—C₁-C₁₂-alkyl-, —(C═O)—C₁-C₁₂-alkenyl-, —(C═O)—C₁-C₁₂-alkynyl-, —(C═O)—C₁-C₁₂-alkylamino-, —(C═O)—C₁-C₁₂-alkenylamino-, —(C═O)—C₁-C₁₂-alkynylamino-, —(C═O)—C₁-C₁₂-alkylamido-, —(C═O)—C₁-C₁₂-alkenylamido-, —(C═O)—C₁-C₁₂-alkynylamido-, —(C═O)—C₁-C₁₂-alkylamido-C₁-C₁₂-alkyl/alkenyl/alkynyl-, —(C═O)—C₁-C₁₂-alkenylamido-C₁-C₁₂-alkyl/alkenyl/alkynyl-, —(C═O)—C₁-C₁₂-alkynylamido-C₁-C₁₂-alkyl/alkenyl/alkynyl-;

and salts thereof.

In one version of the compounds, R⁵ is selected from the group consisting of: hydrogen, alkyl, alkoxy, halo, haloalkyl, hydroxy, hydroxyalkyl, aryl, alkyl-substituted aryl, alkoxy-substituted aryl, and halo-substituted aryl.

In another version of the compounds, R⁶ is selected from the group consisting of hydrogen, alkyl, alkoxy, halo, haloalkyl, hydroxy, hydroxyalkyl, aryl, alkyl-substituted aryl, alkoxy-substituted aryl, and halo-substituted aryl. R⁶ may also be selected from a sub-set of these groups, such as hydrogen, alkyl, alkoxy, halo, haloalkyl, hydroxy, and hydroxyalkyl; or hydrogen, halo, and alkyl.

Alternatively, R⁵ may be selected from the group consisting of hydrogen, alkyl, alkoxy, halo, haloalkyl, hydroxy, and hydroxyalkyl. Or R⁵ may be selected from the group consisting of aryl, alkyl-substituted aryl, alkoxy-substituted aryl, and halo-substituted aryl.

Specific compounds disclosed herein are those wherein the dashed line attached to X is a single bond; X is —(C═O)—; Z is absent; and the dashed line attached to Z is absent.

Additional specific compounds disclosed herein are those wherein the dashed line attached to X is a single bond; X is —(CH₂)—; Z is absent; and the dashed line attached to Z is absent.

Still other compounds specifically disclosed herein are those wherein the dashed line attached to X; X is hydrogen; the dashed line attached to Z is a single bond; and Z is hydrogen.

Other compounds falling within the disclosure include those wherein one of R¹ or R³ is selected from the group consisting of aryl, alkyl-substituted aryl, alkoxy-substituted aryl, and halo-substituted aryl. Additionally included are those compounds wherein one of R² or R⁴ is selected from the group consisting of aryl, alkyl-substituted aryl, alkoxy-substituted aryl, and halo-substituted aryl. Also disclosed herein are those compounds wherein one of R¹ or R³ and one of R² or R⁴ are selected from the group consisting of aryl, alkyl-substituted aryl, alkoxy-substituted aryl, and halo-substituted aryl.

Specific compounds disclosed herein include those selected from the group consisting of:

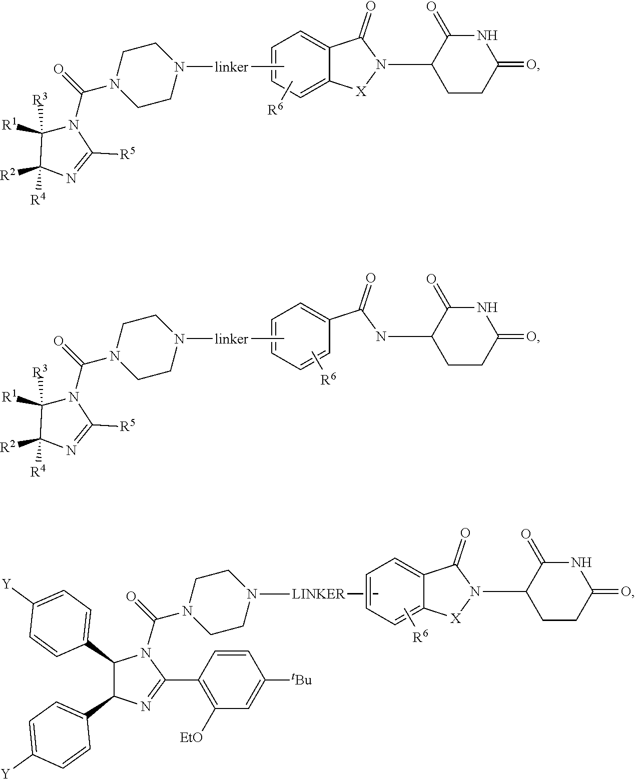

wherein R¹ and R² are not hydrogen, and R³ and R⁴ are hydrogen; and each Y is independently selected from the group consisting of hydrogen, halogen, or C₁-C₆-alkyl.

The compounds disclosed herein may also be selected from the group consisting of:

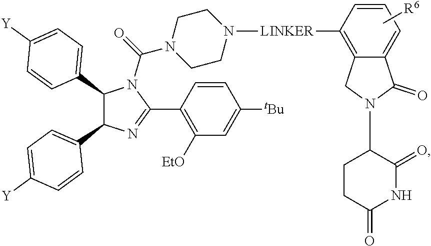

wherein Y and R⁶ are as described previously.

Also disclosed herein are pharmaceutical composition comprising an amount of one or more compounds as recited above, in combination with a pharmaceutically suitable carrier.

Disclosed herein is a method to inhibit neoplastic cell growth, the method comprising contacting a cell with a neoplastic cell growth-inhibiting amount of one or more compounds as described herein. This may include administering to a subject a neoplastic cell growth inhibiting-effective amount of one or more compounds as recited herein. The subject may be a mammalian subject, including a human subject.

The compounds may have the formula:

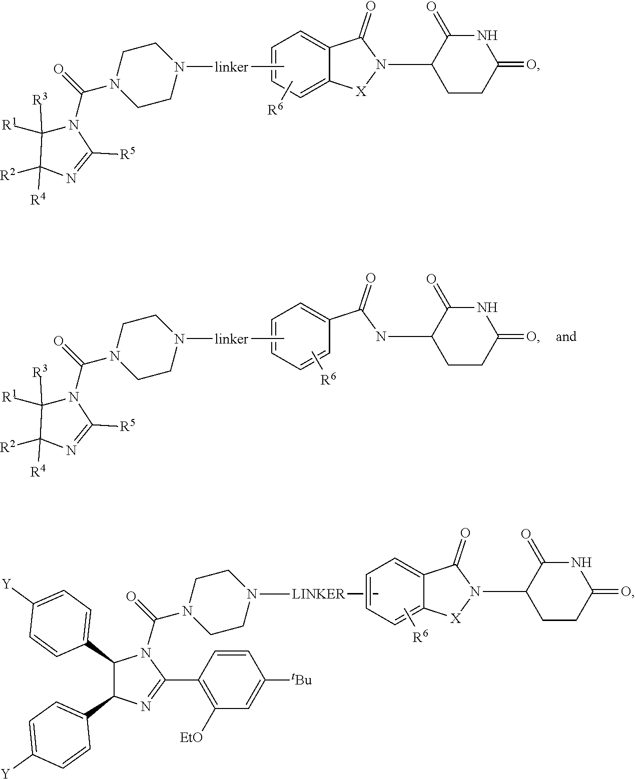

wherein each R¹, R², R³, R⁴, R⁵, and R⁶ are independently hydrogen or a substituent as defined below:

each Y is independently hydrogen, halogen, or C₁-C₆-alkyl; and

X is —(C═O)— or —CH₂—;

“Linker” is selected from the group consisting of a single bond, a C₁-C₁₂-alkylene, akenylene, or akynylene, —(C═O)—C₁-C₁₂-alkyl-, —(C═O)—C₁-C₁₂-alkenyl-, —(C═O)—C₁-C₁₂-alkynyl-, —(C═O)—C₁-C₁₂-alkylamino-, —(C═O)—C₁-C₁₂-alkenylamino-, —(C═O)—C₁-C₁₂-alkynylamino-, —(C═O)—C₁-C₁₂-alkylamido-, —(C═O)—C₁-C₁₂-alkenylamido-, —(C═O)—C₁-C₁₂-alkynylamido-, —(C═O)—C₁-C₁₂-alkylamido-C₁-C₁₂-alkyl/alkenyl/alkynyl-, —(C═O)—C₁-C₁₂-alkenylamido-C₁-C₁₂-alkyl/alkenyl/alkynyl-, —(C═O)—C₁-C₁₂-alkynylamido-C₁-C₁₂-alkyl/alkenyl/alkynyl-;

and salts thereof.

The compounds may have the specific stereo chemistry shown in the following formulas:

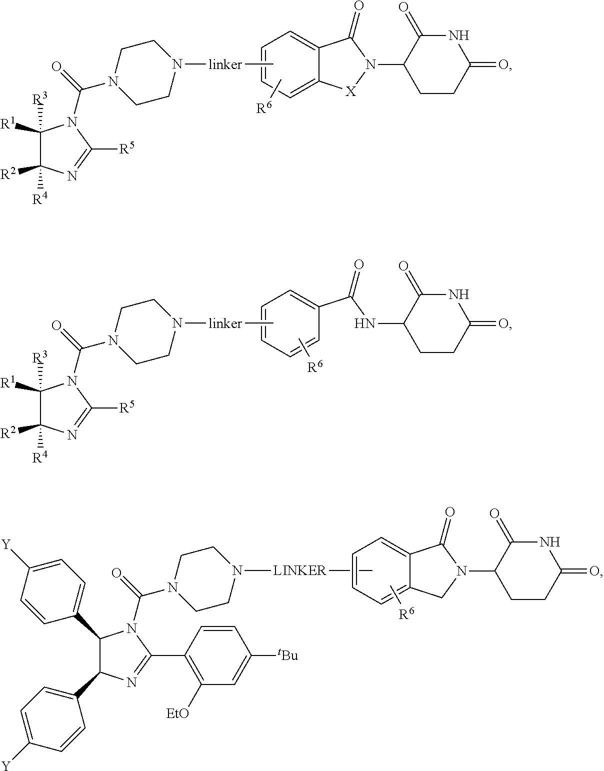

In one version of the compounds, the two moieties are linked as follows:

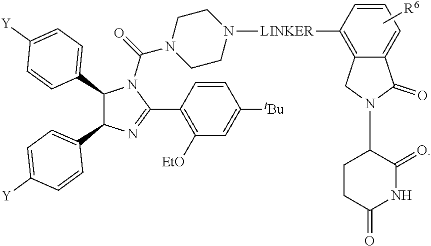

In other versions of the compounds, each Y substituent is chlorine.

Specific compounds disclosed herein include:

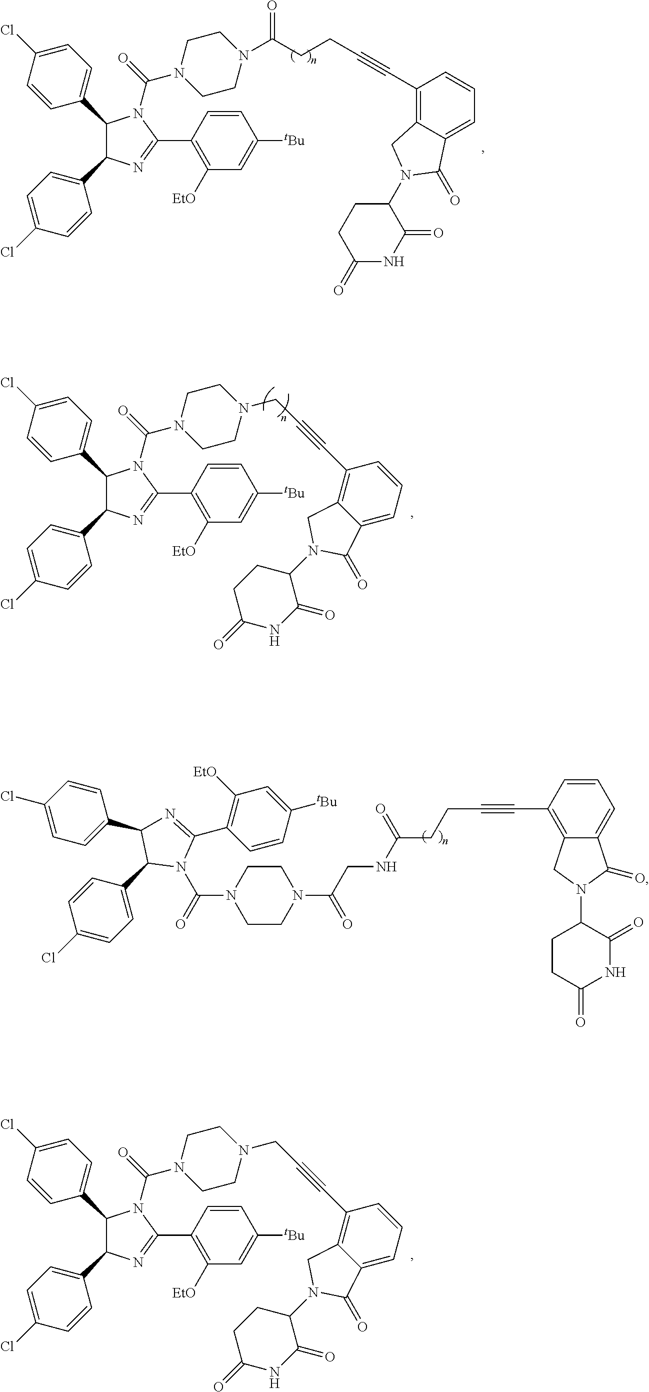

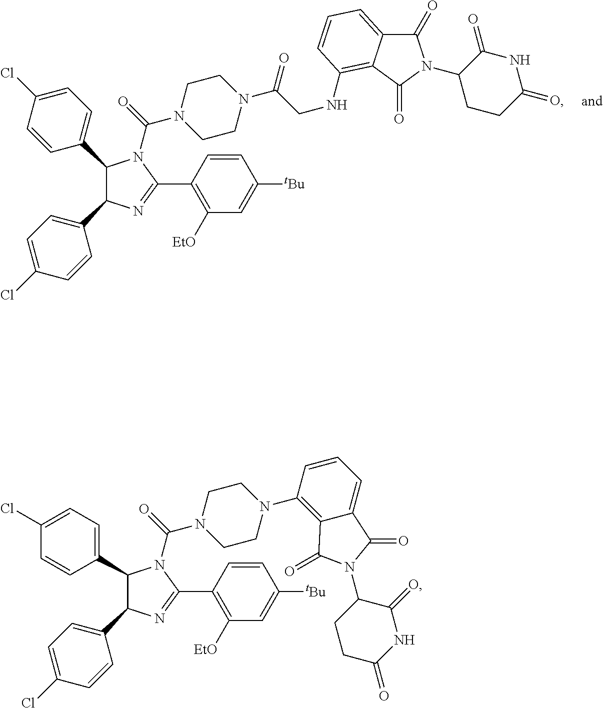 [SMILES: CCOc1cc(C(C)(C)C)ccc1C1=N[C@@H](c2ccc(Cl)cc2)[C@@H](c2ccc(Cl)cc2)N1C(=O)N1CCN(C(=O)CNc2cccc3c2C(=O)N(C2CCC(=O)NC2=O)C3=O)CC1 ; CCOc1cc(C(C)(C)C)ccc1C1=N[C@@H](c2ccc(Cl)cc2)[C@@H](c2ccc(Cl)cc2)N1C(=O)N1CCN(c2cccc3c2C(=O)N(C2CCC(=O)NC2=O)C3=O)CC1]

wherein “n” is an integer of from 1 to 3 (and salts thereof).

Also disclosed herein is a method to inhibit neoplastic cell growth, the method comprising contacting a cell with one or more compounds as disclosed herein. The method includes administering to a subject (e.g., mammals, including humans) a neoplastic cell growth inhibiting-effective amount of one or more compounds disclosed herein.

Also disclosed herein are pharmaceutical composition comprising an amount of one or more compounds as disclosed herein in combination with a pharmaceutically suitable carrier.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a prior art, high-resolution co-crystal structure of the interaction between MDM2 and p53.

FIG. 1B shows four representative, prior art MDM2 inhibitors currently in clinical trials.

FIG. 2A is a schematic representation of proteolysis-targeting chimera (PROTAC) technology.

FIG. 2B is a co-crystal structure of nutlin derivative RG7112 bound to the MDM2 binding site (PDB code: 4IPF).

FIG. 2C depicts the current synthetic strategy to construct MDM2 degraders.

FIG. 3 shows the synthesis of the MDM2 degraders disclosed herein. Reagents and conditions: (a) triphosgene, DIPEA, DCM, N-Boc-piperazine, 74%; (b) HATU, DIPEA, carboxylic acid, DMF, rt, 83%-98%; (c) 3-(4-bromo-1-oxoisoindolin-2-yl)piperidine-2,6-dione (2), Pd(PPh₃)₂Cl₂, CuI, Et₃N, DMF, 70° C., overnight, 11%-39%; (d) alkynyl bromide or iodide, K₂CO₃, THF, reflux, 66%-83%; (e) TFA, DCM, 90%; (f) propargyl alcohol, Pd(PPh₃)₂Cl₂, CuI, Et₃N, DMF, 70° C., overnight, 81%; (g) SO₂Cl₂, Et₃N, DCM, 64%; (h) 1, K₂CO₃, acetone, NaI, reflux, 68%; (i) 2-(2,6-dioxopiperidin-3-yl)-4-fluoroisoindoline-1,3-dione (2′), DMF, DIPEA, 70° C., 23%-27%.

FIG. 4 shows enantiomers of MDM2 ligands, including the optically pure compound (4S, 5R)-1.

FIG. 5 shows the synthesis of optically pure MDM2 ligands and degraders. Reagents and conditions: (a) 4-(tert-butyl)-2-ethoxybenzoic acid, EDC, DMAP, DCM, 96%; (b) TFA, DCM, 92%; (c) CDI, DCM, N-Boc-piperazine, 84%; (d) triphenylphosphine oxide, Tf₂O, DCM, 97%; (e) 3-(4-(3-chloroprop-1-yn-1-yl)-1-oxoisoindolin-2-yl)piperidine-2,6-dione (24), K₂CO₃, acetone, NaI, reflux, 59%; (f) K₂CO₃, THF, reflux, 61%-72%; (g) 3-(4-bromo-1-oxoisoindolin-2-yl)piperidine-2,6-dione (2), Pd(PPh₃)₂Cl₂, CuI, Et₃N, DMF, 70° C., 10%-28%.

FIG. 6A is a histogram presenting the anti-proliferation effect of the racemic PROTACs tested (compound numbers are shown in X-axis.) on RS4;11 cells. Cell viability was evaluated at 100 nM concentration of each compound using the MTT assay.

FIG. 6B is a histogram showing the anti-proliferation effect of the chiral PROTACs tested (compound numbers are shown in X-axis.) on the RS4;11 cells. Cell viability was evaluated at 100 nM concentration of each compound using the MTT assay;

FIG. 6C is a graph comparing the anti-proliferation effect of degrader 20 and its parent MDM2 ligand 1;

FIG. 6D is a graph comparing the anti-proliferation effect of degrader 32 and its parent MDM2 ligand (4S, 5R)-1.

FIG. 7A is an immunoblot of MDM2 and p53 following treatment with DMSO or the indicated MDM2 degraders.

FIG. 7B is an immunoblot of MDM2, p53 and cleaved poly(ADP-ribose) polymerase (“PARP”) following incubation with DMSO or 32 for the indicated time.

FIG. 7C is an immunoblot of MDM2 and p53 following 24 h incubation with DMSO or the indicated concentrations of compound 32.

FIG. 7D is a band intensity graph of the immunoblot shown in FIG. 7C. The curve was calculated using the public domain “Image J” software (available for download from the U.S. National Institutes of Health at imagej.nih.gov/ij/download.html) and plotted and fitted using GraphPad Prism-brand software (GraphPad Software 2365 Northside Dr., Suite 560, San Diego, Calif. 92108).

FIG. 7E is an immunoblot showing that degradation is dependent on cereblon (CRBN), MDM2, and the proteasome. The cells were pre-treated with proteasome inhibitor MG132 (5 μM), MDM2 ligand (4S, 5R)-1 (5 μM), or the CRBN ligand lenalidomide (5 μM), for 2 h, followed by treatment with DMSO or compound 32 for 4 h.

FIG. 7F is an immunoblot showing that the downregulation of MDM2 by a PROTAC is reversible. The cells were treated with compound 32 for 12 h, then the media was replaced with fresh medium and the cells were washed thoroughly to remove residual 32. Immunoblot analysis of MDM2 level was conducted at the indicated times.

FIGS. 8A, 8B, and 8C are quantitative RT-PCR analysis of mRNA levels of MDM2 (FIG. 8A), p53 (FIG. 8B) and CDKN1A (p21) (FIG. 8C) in RS4;11 cells. In all three figures, cells were treated with DMSO, MDM2 ligand (4S,5R)-1 (1 PM), or MDM2 degrader 32 at 30 nM and 100 nM for 6 h. mRNA level of MDM2, TP53, and p21 were analyzed. The result was normalized to GAPDH and statistically analyzed in one-way ANOVA, ***P<0.001, ****P<0.0001.

FIG. 9A is an immunoblot showing that degradation of MDM2 induced the up-regulation of p21 and the cleavage of PARP and Caspase-3 in RS4;11 cells.

FIG. 9B presents flow cytometry analysis of the apoptosis induction by 32 in RS4;11 cells. Cells were treated with MDM2 ligand (4S,5R)-1 or degrader 32 at the indicated concentrations for 24 h. Apoptosis was assessed by flow cytometry using Annexin V and propidium iodide dual staining.

FIG. 9C is a bar graph showing quantification of apoptotic cells based on the flow cytometry experiments from FIG. 9B.

### DETAILED DESCRIPTION

### Abbreviations and Definitions

CDI=carbonyldiimidazole. CDKN1A=cyclin-dependent kinase inhibitor 1A protein (also known as p21). DCM=dichloromethane. DIPEA=N,N-diisopropylethylamine. DMAP=4-dimethylaminopyridine. DMF=dimethylformamide. EDC=1-ethyl-3-(3-dimethylaminopropyl)carbodiimide. ee=enantiomeric excess. FBS=fetal bovine serum. FITC=fluorescein isothiocyanate. HATU=hexafluorophosphate azabenzotriazole tetramethyl uronium; systematic name 1-[bis(dimethylamino)methylene]-1H-1,2,3-triazolo[4,5-b]pyridinium 3-oxide hexafluorophosphate. HEPES=(4-(2-hydroxyethyl)-1-piperazineethanesulfonic acid). MDM2=murine double minute 2 protein. PARP=poly(ADP-ribose) polymerase. PBS=phosphate-buffered saline. PI=propidium iodide. PROTAC=proteolysis-targeting chimera. PVDF=polyvinylidene fluoride. SDS-PAGE=sodium dodecyl sulfate-polyacrylamide gel electrophoresis. TFA=tetrafluoroacetic acid. THF=tetrahydrofuran.

The “MTT assay” is a colorimetric assay for assessing cellular metabolic activity. NAD(P)H-dependent cellular oxidoreductase enzymes reflect the number of viable cells present. These enzymes are capable of reducing the tetrazolium dye 3-(4,5-dimethylthiazol-2-yl)-2,5-diphenyltetrazolium bromide (“MTT”) to its insoluble formazan, which has a purple color and whose concentration is then measured spectrophotometrically. The MTT assay is conventional and well known. See, for example, Stockert et al. (2018) “Tetrazolium salts and formazan products in Cell Biology: Viability assessment, fluorescence imaging, and labeling perspectives,” Acta Histochemica 120: 159-167. As used herein, “MTT assay” refers broadly to the MTT assay itself and to the well-known variations of it that use structurally related tetrazolium salts (e.g., XTT, MTS, etc.).

“p53” is used herein to refer collectively to any and all isoforms of tumor protein p53, which is also known as “cellular tumor antigen p53” (UniProt name), “phosphoprotein p53,” “tumor suppressor p53,” “antigen NY-CO-13,” and “transformation-related protein 53” (TRP53). P53 proteins are encoded by homologous genes in a host organisms, including TP53 in humans and Trp53 in mice.

RS4;11 cells are human acute lymphoblastic leukemia cells, Accession No. ATCC® CRL-1873, available commercially from the American Type Culture Collection (ATCC), 10801 University Boulevard, Manassas, Va. 20110 USA. (“ATTC” is a registered trademark of the American Type Culture Collection.).

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. That is, unless specifically stated to the contrary, “a” and “an” mean “one or more.” The phrase “one or more” is readily understood by one of skill in the art, particularly when read in context of its usage. For example, “one or more” substituents on a phenyl ring designates one to five substituents.

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in synthetic organic chemistry.

The term “contacting” refers to the act of touching, making contact, or of bringing to immediate or close proximity, including at the molecular level, for example, to bring about a chemical reaction, or a physical change, e.g., in a solution or in a reaction mixture.

An “effective amount” refers to an amount of a chemical or reagent effective to facilitate a chemical reaction between two or more reaction components, and/or to bring about a recited effect. Thus, an “effective amount” generally means an amount that provides the desired effect.

The term “solvent” refers to any liquid that can dissolve a compound to form a solution. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (sec or tert) butanol, and the like. Aprotic solvents that can be used in the method include, but are not limited to perfluorohexane, α,α,α-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

The term “alkyl” refers to a branched or unbranched carbon chain having, for example, about 1-20 carbon atoms, and often 1-12, 1-10, 1-8, 1-6, or 1-4 carbons. Examples include, but are not limited to, methyl, ethyl, 1-propyl, 2-propyl, 1-butyl, 2-methyl-1-propyl, 2-butyl, 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted. The alkyl can also be optionally partially or fully unsaturated in certain embodiments. As such, the recitation of an alkyl group optionally includes both alkenyl and alkynyl groups. The alkyl can be a monovalent hydrocarbon radical, as described and exemplified above, or it can be a divalent hydrocarbon radical (i.e., an alkylene). In some embodiments, certain alkyl groups can be excluded from a definition. For example, in some embodiments, methyl, ethyl, propyl, butyl, or a combination thereof, can be excluded from a specific definition of alkyl in an embodiment.

The terms “amine” and “amino” refer to both the internal functional group having the structure —(N—R)—, where R may be hydrogen or a substituent (as defined below) and the terminal functional group —NR₂, where the two R groups may be the same or different and are hydrogen or a substituent. “Amine” and “amino” also encompass quaternized salts of these nitrogen-containing functional groups.

The terms “amide and “amido” refer to the functional group having the structures —(C═O)—N(R)— (for an internal amido group), and —(C═O)—NR₂ (for a terminal amido group). Each R may be the same or different and may be hydrogen or a substituent.

The term “substituted” indicates that one or more hydrogen atoms on the group indicated in the expression using “substituted” is replaced with a “substituent”. The number referred to by ‘one or more’ can be apparent from the moiety one which the substituents reside. For example, one or more can refer to, e.g., 1, 2, 3, 4, 5, or 6; in some embodiments 1, 2, or 3; and in other embodiments 1 or 2. The substituent can be one of a selection of indicated groups, or it can be a suitable group known to those of skill in the art, provided that the substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, aroyl, (aryl)alkyl (e.g., benzyl or phenylethyl), heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl(alkyl)amine, and cyano. Additionally, suitable substituent groups can be, e.g., —X, —R, —O—, —OR—, —SR—, —S—, —NR₂, —NR₃, ═NR, —CX₃, —CN, —OCN, —SCN, —N═C═O, —NCS, —NO, —NO2, ═N₂, —N₃, —NC(═O)R, —C(═O)R, —C(═O)NRR, —S(═O)₂O—, —S(═O)₂OH, —S(═O)₂R, —OS(═O)₂OR, —S(═O)₂NR, —S(═O)R, —OP(═O)O₂RR, —P(═O)O₂RR, —P(═O)(O—)₂, —P(═O)(OH)₂, —C(═O)R, —C(═O)X, —C(S)R, —C(O)OR, —C(O)O—, —C(S)OR, —C(O)SR, —C(S)SR, —C(O)NRR, —C(S)NRR, or C(NR)NRR, where each X is independently a halogen (“halo”): F, Cl, Br, or I; and each R is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for substituents on the substituted group.

A protecting group is any chemical moiety capable of selective addition to and removal from a reactive site to allow manipulation of a chemical entity at sites other than the reactive site. Many protecting groups are known in the art. A large number of protecting groups and corresponding chemical cleavage reactions are described in “Protective Groups in Organic Synthesis,” Theodora W. Greene (John Wiley & Sons, Inc., New York, 1991, ISBN 0-471-62301-6) (“Greene”, which is incorporated herein by reference). See also the 5^{th} edition of this same work, published under the title “Greene's Protective Groups in Organic Synthesis,” ISBN-13: 978-1118057483, ©2014, John Wiley & Sons, Inc. Greene describes many nitrogen protecting groups, for example, amide-forming groups. In particular, see Chapter 1, Protecting Groups: An Overview, Chapter 2, Hydroxyl Protecting Groups, Chapter 4, Carboxyl Protecting Groups, and Chapter 5, Carbonyl Protecting Groups. For additional information on protecting groups, see also Kocienski, Philip J. “Protecting Groups,” (Georg Thieme Verlag Stuttgart, New York, 1994), which is incorporated herein by reference. Typical nitrogen protecting groups described in Greene include benzyl ethers, silyl ethers, esters including sulfonic acid esters, carbonates, sulfates, and sulfonates. For example, suitable nitrogen protecting groups include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyl-diphenylmethyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters (formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate(mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methylphenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)benzoate, p-poly-benzoate, a-naphthoate, nitrate, alkyl N,N,N′,N′-tetramethyl-phosphorodiamidate, n-phenylcarbamate, borate, 2,4-dinitrophenylsulfenate); or sulfonates (methanesulfonate(mesylate), benzenesulfonate, benzylsulfonate, tosylate, or triflate).

The more common of the amine-protecting groups have trivial abbreviations that are widely used in the literature and include: carbobenzyloxy (Cbz) group (removed by hydrogenolysis), p-methoxybenzyl carbonyl (Moz or MeOZ) group (removed by hydrogenolysis), tert-butyloxycarbonyl (BOC) group (common in solid phase peptide synthesis; removed by concentrated strong acid (such as HCl or CF₃COOH), or by heating to >80° C., 9-fluorenylmethyloxycarbonyl (FMOC) group (also common in solid phase peptide synthesis; removed by base, such as piperidine), acetyl (Ac) group (removed by treatment with a base), benzoyl (Bz) group (removed by treatment with a base), benzyl (Bn) group (removed by hydrogenolysis), carbamate group (removed by acid and mild heating), p-methoxybenzyl (PMB) (removed by hydrogenolysis), 3,4-dimethoxybenzyl (DMPM) (removed by hydrogenolysis), p-methoxyphenyl (PMP) group (removed by ammonium cerium(IV) nitrate (CAN)), tosyl (Ts) group (removed by concentrated acid and strong reducing agents), sulfonamide groups (Nosyl & Nps; removed by samarium iodide, tributyltin hydride.

A “pharmaceutically-suitable salt” is any acid or base addition salt whose counter-ions are non toxic to a patient (including a veterinary patient) in pharmaceutical doses of the salts, so that the beneficial pharmacological effects inherent in the free base or free acid are not vitiated by side effects ascribable to the counter-ions. A host of pharmaceutically-suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically-suitable salt by ion exchange procedures. Pharmaceutically-suitable salts include, without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, methylene bis-b-hydroxynaphthoates, gentisates, isethionates, di-p-toluoyltartrates, methane sulphonates, ethanesulphonates, benzenesulphonates, p-toluenesulphonates, cyclohexylsulphamates, quinates, and the like. Base addition salts include those derived from alkali or alkaline earth metal bases or conventional organic bases, such as triethylamine, pyridine, piperidine, morpholine, N methylmorpholine, and the like.

### The Approach:

Due to the significant anti-cancer effect of MDM2 inhibitors as discussed before, their potential application for the treatment of fragile X-syndrome[52], and recent interest in targeted protein degradation, [45, 53], we set out to develop novel MDM2 degraders based in part on derivatizing the nutlin core:

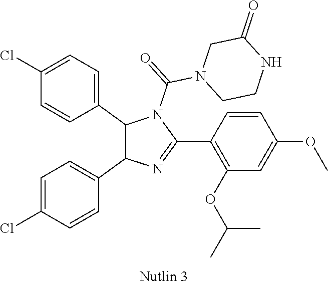 [SMILES: COc1ccc(C2=NC(c3ccc(Cl)cc3)C(c3ccc(Cl)cc3)N2C(=O)N2CCNC(=O)C2)c(OC(C)C)c1]

### (±)-4-[4,5-Bis(4-chlorophenyl)-2-(2-isopropoxy-4-methoxy-phenyl)-4,5-dihydro-imidazole-1l-carbonyl]-piperazin-2-one

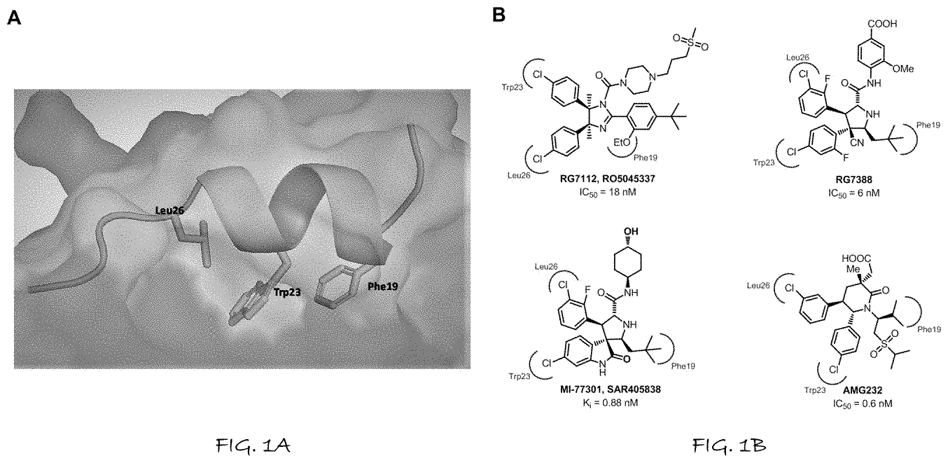

The MDM2 degraders disclosed herein comprise three main components: a MDM2 binder or inhibitor, which binds MDM2; a ligand to recruit the E3 ligase; and a linker that connects these two parts. Nutlin derivative RG7112, a highly potent MDM2 inhibitor (IC₅₀=18 nM), is the first small molecule MDM2 inhibitor in clinical trials[20] (see FIG. 1B):

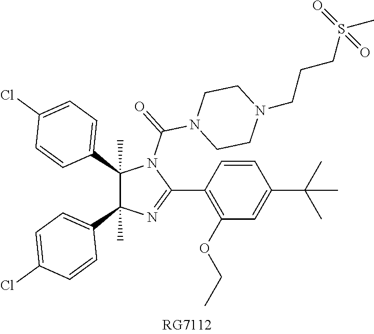 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

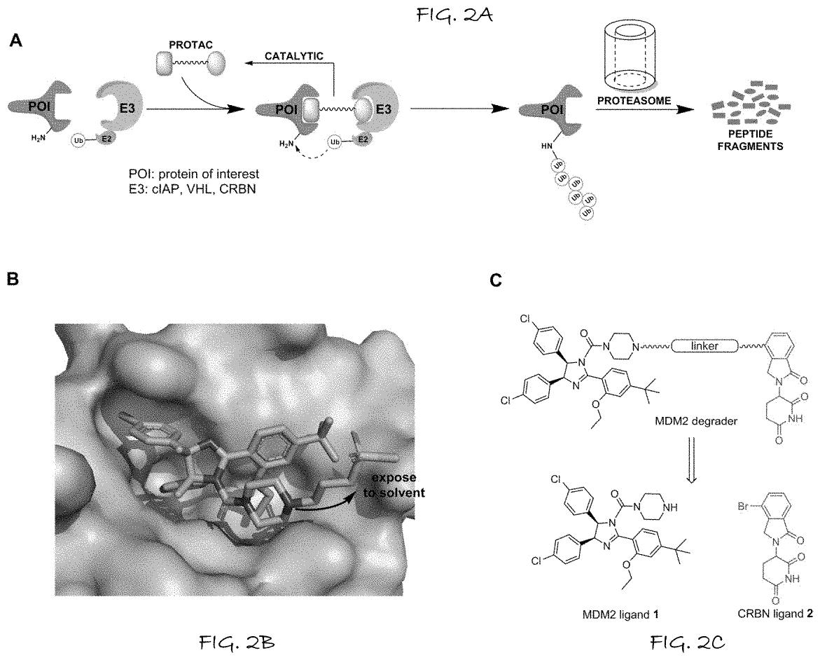

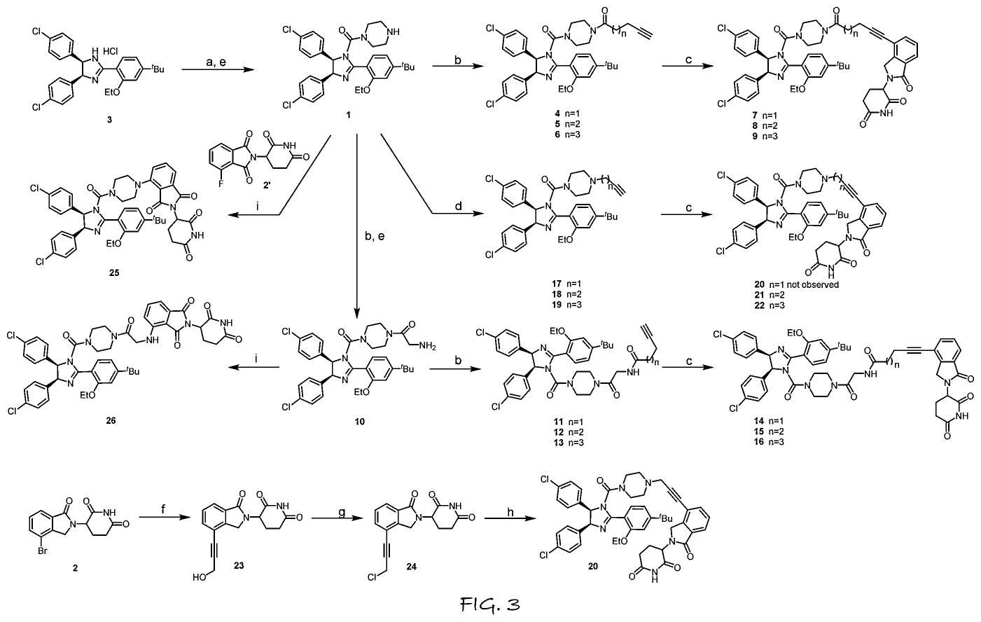

It disrupts the MDM2-p53 interaction in p53 wild-type cancerous cells. The co-crystal structure of RG7112 and MDM2 (depicted in FIG. 2B) shows that the two 4-chlorophenyl rings and the ethoxy group occupy the Leu26, Trp23 and Phe19 pockets, respectively. The piperazine ring is exposed to the solvent. Thus it was decided to place the linker that connects to the E3 ligase ligand at the piperazine ring. See FIG. 2C, top structure. With this information in mind, a simpler analogue of RG7112, ligand 1, was designed to construct MDM2 degraders (see FIG. 2C, bottom structure):

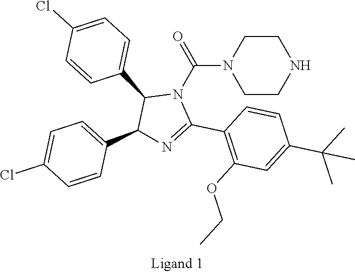 [SMILES: CCOc1cc(C(C)(C)C)ccc1C1=N[C@@H](c2ccc(Cl)cc2)[C@@H](c2ccc(Cl)cc2)N1C(=O)N1CCNCC1]

Here, novel MDM2 degraders were designed by chemically appending MDM2 ligand 1 to CRL4^{CRBN} E3 ligase ligand 2. See FIG. 2C.

The synthesis of degraders is outlined in FIG. 3. Cis-imidazoline 3 was prepared according to the reported procedures[56, 57]. Through the formation of urea followed by acid-mediated deprotection, 3 was converted to MDM2 ligand 1. E3 ligase CRBN ligand 2[39] (bottom left of FIG. 3) was then attached to MDM2 ligand 1 by various linkers. As outlined in FIG. 3, acylation of the secondary amine in 1 gave intermediates 4-6 and 11-13, which were appended to ligand 2 by the Sonogashira cross-coupling reaction to afford 7-9 and 14-16. Compounds 17-19 were prepared by the direct N-alkylation of MDM2 ligand 1. A Sonogashira cross-coupling reaction then yielded products 21 and 22. An alternative method was used to produce degrader 20, which involved the alkylation of 1 by chloride 24, derived from 2 in two steps. Two “linkerless” degraders 25 and 26 were prepared as well by the alkylation of 1 or 10 with 2-(2,6-dioxopiperidin-3-yl)-4-fluoroisoindoline-1,3-dione 2′ in the presence of DIPEA. This library of potential MDM2 degraders covered a broad range of linker lengths and types.

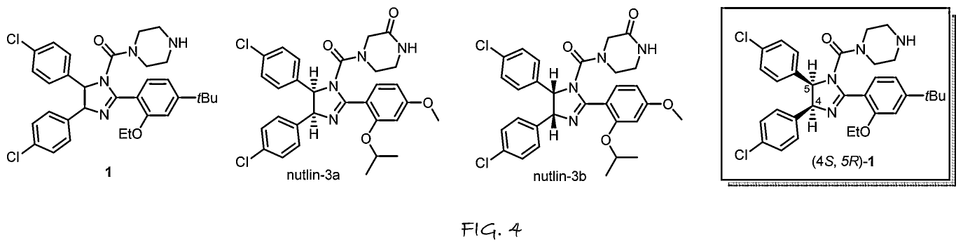

MDM2 ligand 1 is racemic. See FIG. 4. The activity of levorotatory enantiomer nutlin-3a is approximately 120-fold more potent than the dextrorotatory isomer nutlin-3b. We hypothesized that an optically pure ligand (4S, 5R)-1 might further increase the efficacy of the designed bifunctional degraders. That said, the disclosure and claims cover any stereoisomer of 1, in a racemic mixture or an any range of enantiomeric excess toward one of the stereoisomers.

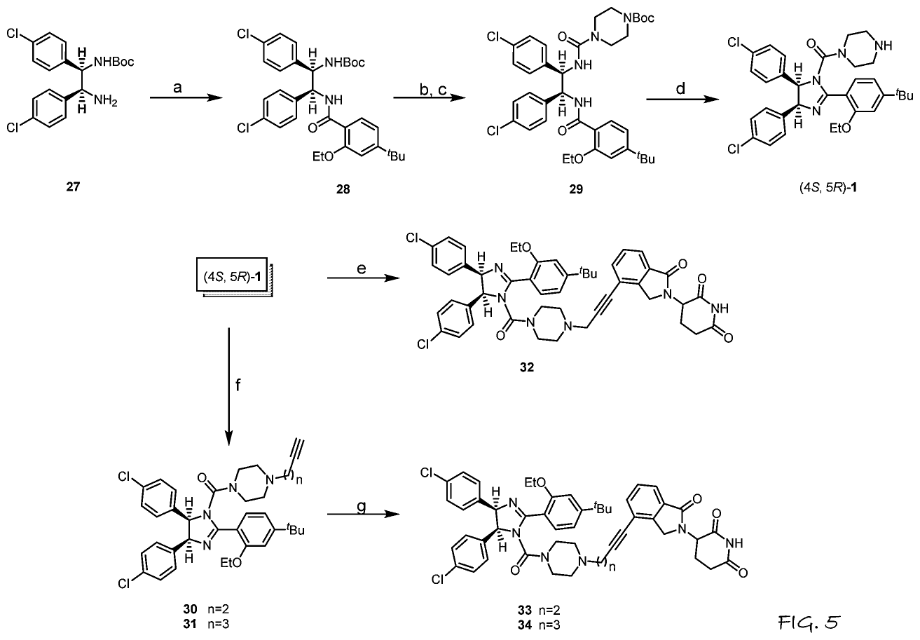

The catalytic asymmetric synthesis of (4S, 5R)-1 is outlined in FIG. 5. Using Johnston's catalytic aza-Henry reaction [58, 59], followed by reduction of the nitro group, mono-protected cis-stilbene diamine 27 was synthesized enantioselectively (99% ee). The acylation of 27 by benzoic acid then gave 28, which was subjected to tert-butyl carbamate deprotection and followed by CDI-mediated urea formation, affording product 29. Cyclization of 29 using a phosphonium anhydride, formed by the combination of triphenylphosphineoxide and triflic anhydride (Hendrickson's reagent), gave the optically pure MDM2 ligand (4S, 5R)-1. Using 1, three additional degraders 32, 33 and 34 were prepared.

### Results:

Screening of MDM2 Degraders in an Anti-Proliferation Assay:

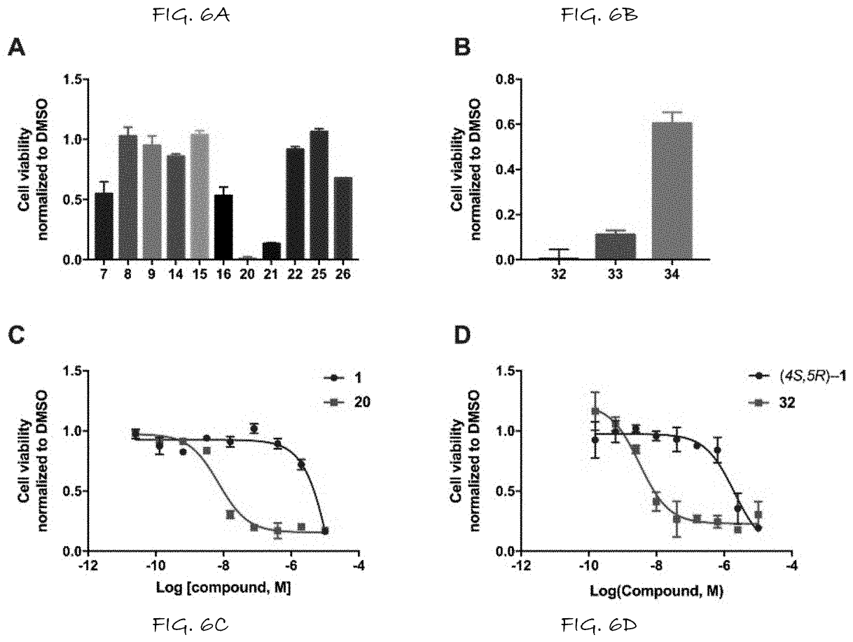

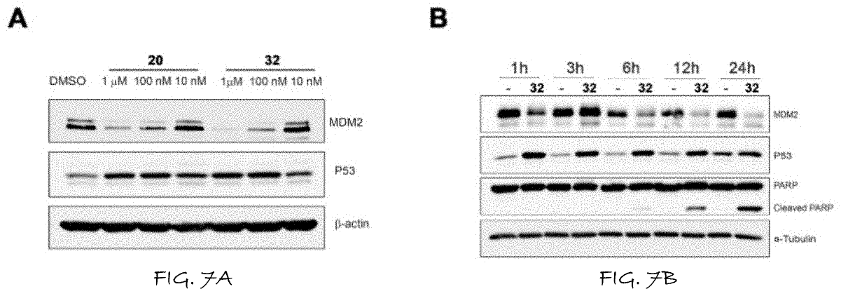

All degraders were evaluated by MTT assay on different cell lines (e.g. SJSA-1, MCF-7, MM1.S, HepG2, A549, A431 and RS4;11). Among them, the RS4;11 cell line was the most responsive to the degraders. Degrader 20, with the shortest linker, was the most potent inhibitor of RS4;11 (compounds tested at 100 nM concentration) (FIG. 6A). As previously mentioned, nutlin-3a is 120-fold more potent that its enantiomer nutlin-3b for the inhibition of cell growth. Thus, the optically pure MDM2 ligands disclosed herein were investigated to see if one might have increased anti-proliferation potency as compared to the other. Compounds 32, 33 and 34 were then synthesized and evaluated for their anti-proliferation activity in RS4;11 cells at 100 nM (FIG. 6B). Similar to the racemic series, compound 32 with the shortest linker was the most potent compound as compared to 33 and 34. It strongly inhibits the growth of RS4;11 cells with an IC₅₀ value of 3.2 nM, more potent than the corresponding racemic compound 20 (IC₅₀=7.2 nM). Degrader 20 (IC₅₀=7.2 nM) is significantly more potent than its parent MDM2 inhibitor 1 (FIG. 6C). As shown in FIG. 6C and FIG. 6D, the anti-proliferation effect of degraders 20 and 32 are nearly 1,000-fold more potent than their parent MDM2 inhibitors 1 and (4S, 5R)-1, respectively, demonstrating the outperformance of event-driven degradation over occupancy-driven inhibition[60, 61].

Degrader 32 Effectively Induces MDM2 Degradation:

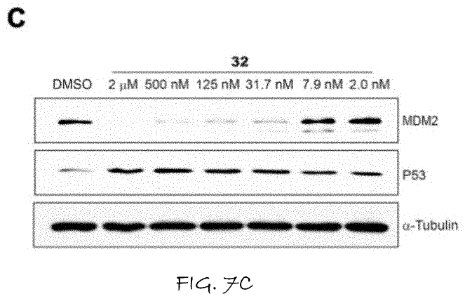

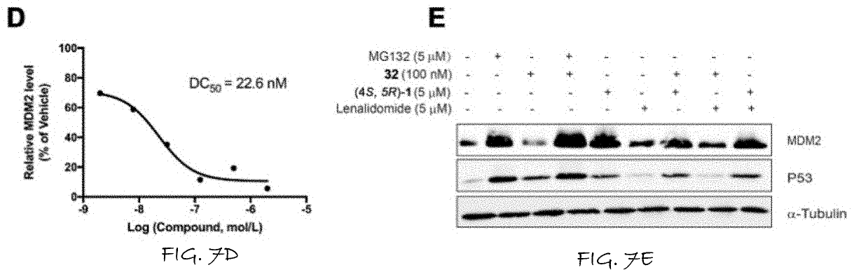

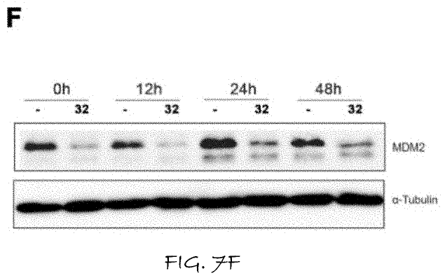

To evaluate the extent of MDM2 degradation by synthesized PROTACs, Western blot was used to analyze the protein level of MDM2 in RS4;11 leukemia cells. It was found that PROTACs 7, 15, 20, 32 could all degrade significant amounts of MDM2 (data not shown). Because degrader 32 has the most potent anti-proliferation activity, we then further characterized the mechanism of action of degrader 32. We started by comparing the degradation effect of 20 and 32, which differ only in the chirality of the MDM2 ligand. Degrader 20 features racemic ligand 1, while degrader 32 features chiral ligand (4S, 5R)-1. Using 100 nM of both 20 and 32, significant degradation of MDM2 was observed, but 32 was superior under identical conditions. During MDM2 degradation, p53 simultaneously accumulates (FIG. 7A). The time-dependence of MDM2 degradation by 32 is shown in FIG. 7B. The degradation of MDM2 was observed as early as 1 h into the treatment and the cleaved PARP was observed at 6 h treatment. Furthermore, a dose-dependent experiment was performed by treating cells with different concentrations of 32, and the degradation rate of MDM2 was directly proportional to the concentration of 32 (FIG. 7C). The maximal level of degradation (Dₘₐₓ=90%) was observed at 100 nM, while the concentration at which 50% degradation was detected (DC₅₀) was 23 nM (FIG. 7D). All of these data clearly indicate that compound 32 is a very effective MDM2 degrader.

To further verify the involvement of MDM2, CRL4^{CRBN} E3 ligase and the proteasome in the degradation of MDM2 promoted by compound 32, co-treatments with various inhibitors were performed (FIG. 7E). The degradation of MDM2 by PROTAC 32 at 100 nM was significantly reduced by the addition of 5 μM of MDM2 ligand (4S, 5R)-1 or 5 μM of CRBN ligand lenalidomide. This is consistent with the degrader-induced formation of a ternary complex, which can be disrupted by external ligands that can bind to either MDM2 or CRBN. To confirm that the degradation of MDM2 is through the proteasome system, cells were co-incubated with proteasome inhibitor MG132 (5 PM) and degrader 32. As shown in FIG. 7E, the degradation of MDM2 ceased and 32 acted only as a MDM2 inhibitor, suggesting that the depletion of MDM2 by PROTAC 32 is dependent on the ubiquitin-proteasome system. Finally, a washout experiment was performed to investigate the re-synthesis rate of MDM2 (FIG. 7F). MDM2 levels were increased after the removal of degrader 32, indicating that the downregulation of MDM2 by PROTAC is reversible. MDM2 levels did not return to pre-treatment levels even after 48 hours, indicating a slow re-synthesis rate of MDM2 in RS4;11 cells.

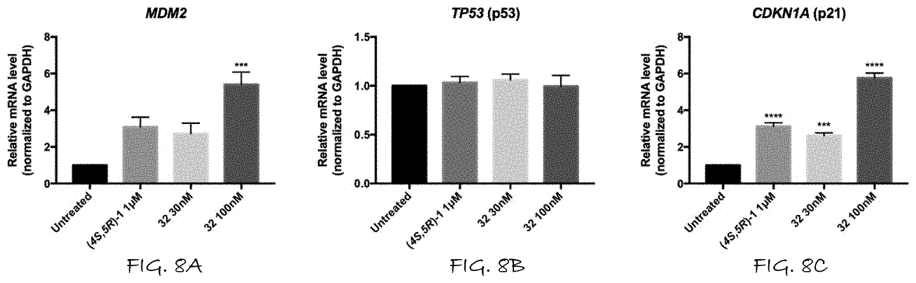

Because depletion of MDM2 can restore the function of p53, we investigated the effect of degrader 32 on the transcriptional activity of p53 in RS4;11 cells by qRT-PCR (FIGS. 8A, 8B, and 8C). Upon treatment of cells with MDM2 ligand (4S, 5R)-1, the targeted genes of p53 are upregulated, including MDM2 (FIG. 8A) and cell cycle regulator gene CDKN1A (p21) (FIG. 8C). Compared to MDM2 ligand (4S, 5R)-1, degrader 32 induces more significant transcriptional upregulation of these p53 targeted genes at 100 nM. Neither MDM2 ligand (4S, 5R)-1 nor degrader 32 has any effect on TP53 in RS4;11 cells (FIG. 8B). Notwithstanding the upregulation of MDM2 mRNA, the efficiency of degrader 32 is assured by significantly reducing MDM2 protein level. Together, the mechanistic investigation confirms that compound 32 acts as a bona fide MDM2 degrader following the mechanism we proposed.

Degrader 32 Initiates the Apoptosis of RS4;11 Cells:

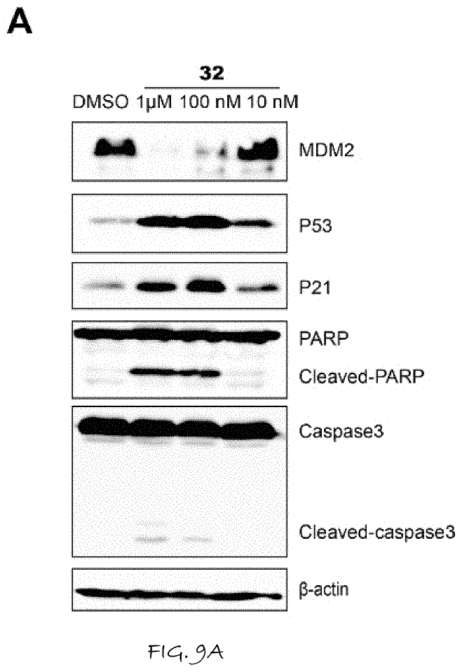

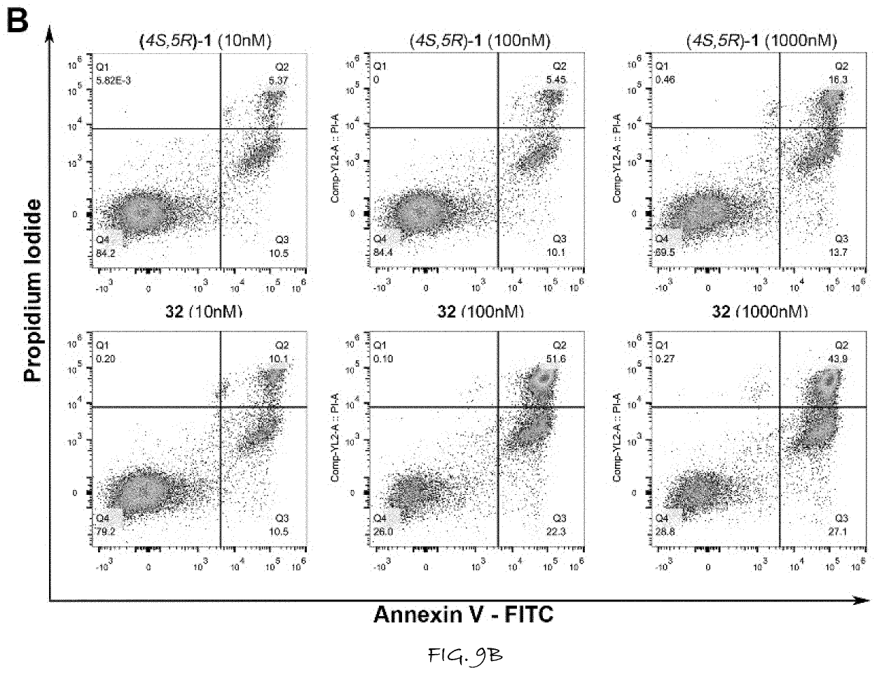

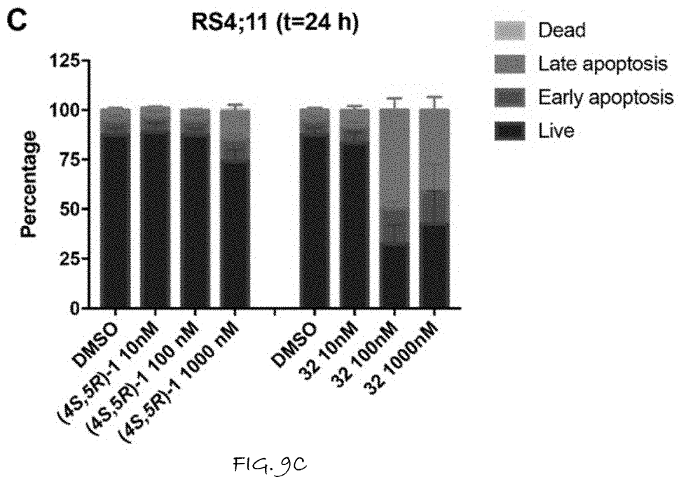

As previously mentioned, degrader 32 acted as a potent MDM2 degrader and inhibited the growth of RS4;11 leukemia cells. To better understand the cellular mechanism that underpins cell viability, we set out to elucidate the consequences of MDM2 degradation by compound 32. The RS4;11 cells were treated with the indicated concentration of 32, as shown in FIG. 9A. At 100 nM or 1 μM of degrader 32, MDM2 depleted and p53 was stabilized and upregulated. A downstream factor of p53, protein p21 functions as a cell cycle regulator. The increased level of p21 indicated the initiation of cell cycle arrest. We then examined the cleavage of PARP and Caspase-3, both of which are critical in cell apoptosis. The cleaved PARP and Caspase-3 were observed under 100 nM or 1 μM concentrations of 32, which indicated the initiation of apoptosis. The dose-dependent apoptosis was further confirmed by flow cytometry analysis (FIG. 9B). A large number of cells were identified in Annexin V-positive quadrants (right top and bottom) when they were treated with 100 nM or 1 μM of degrader 32, which is much more significant than the corresponding MDM2 inhibitor (4S, 5R)-1 (FIGS. 9B and 9C). In summary, the depletion of MDM2 by degrader 32 (100 nM) induces the stabilization of p53, followed by upregulating the level of p21. Moreover, the apoptosis pathway is initiated, which contributes to the high potency of anti-proliferation of degrader 32 for RS4;11 leukemia cells.

### Pharmaceutical Compositions:

Also disclosed herein are pharmaceutical compositions comprising one or more of the compounds disclosed herein and their isotopic forms or a pharmaceutically suitable salt thereof as described herein. More specifically, the pharmaceutical composition may comprise one or more of the compounds described herein (or their salts) in an amount suitable for inhibiting neoplastic cell growth, in combination with a standard, well-known, non-toxic pharmaceutically suitable carrier, adjuvant or vehicle such as, for example, phosphate buffered saline, water, ethanol, polyols, vegetable oils, a wetting agent or an emulsion such as a water/oil emulsion. The composition may be in either a liquid, solid or semi-solid form. For example, the composition may be in the form of a tablet, capsule, ingestible liquid or powder, injectible, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, one or more compounds produced as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and one or more of the anti-neoplastic compounds disclosed herein.

For intravenous administration, the compounds may be incorporated into commercial formulations such as Intralipid©-brand fat emulsions for intravenous injection. (“Intralipid” is a registered trademark of Fresenius Kabi AB, Uppsalla, Sweden.) Where desired, the individual components of the formulations may be provided individually, in kit form, for single or multiple use. A typical intravenous dosage of a representative compound as described herein is from about 0.1 mg to 100 mg daily and is preferably from 0.5 mg to 3.0 mg daily. Dosages above and below these stated ranges are specifically within the scope of the claims.

Possible routes of administration of the pharmaceutical compositions include, for example, enteral (e.g., oral and rectal) and parenteral. For example, a liquid preparation may be administered orally or rectally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect and the medical state of the subject being treated. The dosage of the composition to be administered to the patient may be determined by one of ordinary skill in the medical and/or pharmaceutical arts and depends upon various factors such as weight of the patient, age of the patient, immune status of the patient, etc., and is ultimately at the discretion of the medical professional administering the treatment.

With respect to form, the composition may be, for example, a solution, a dispersion, a suspension, an emulsion or a sterile powder which is then reconstituted. The composition may be administered in a single daily dose or multiple doses.

For a complete discussion of pharmaceutical manufacturing, see “Handbook of Pharmaceutical Manufacturing Formulations,” 2^{nd} Edition, © 2018, CRC Press (Boca Raton, Fla.).

The present disclosure also includes treating neoplastic growth disorders in mammals, including humans, by administering a neoplastic cell growth inhibitory-effective amount of one or more of the compounds described herein. In particular, the compositions of the present invention may be used to treat neoplastic conditions of any and all description, but most notably those mediated or accelerated by HDAC6 enzymes.

It should be noted that the above-described pharmaceutical compositions may be utilized in connection with non-human animals, both domestic and non-domestic, as well as humans.

### Conclusions:

In summary, we designed, synthesized and evaluated a series of PROTAC molecules that can effectively degrade MDM2 oncoprotein in cells, thus stabilizing the tumor suppressor p53. Degrader 32 was identified as the most potent compound for anti-proliferation among the compounds tested. This degrader has one of the shortest linkers between the two ligands among all reported PROTACs. It induced efficient degradation of MDM2 at low nano-molar concentration in RS4;11 leukemia cells carrying wild-type p53. It also inhibits the proliferation of leukemia cells with an IC₅₀ value of 3.2 nM and initiates the apoptosis efficiently. Collectively, degradation of MDM2 by PROTAC molecules could be a promising strategy for the treatment of hematologic cancers.

### Examples

The following examples are included to provide a more complete description of the compounds and methods disclosed and claimed herein. The examples are not intended to limit the scope of the claims in any fashion.

### Compound Synthesis:

Unless otherwise noted, all reagents were purchased from commercial sources and used without further purification. Dry solvents were obtained from a solvent purification system. HRMS were recorded on a Bruker Solarix XR mass spectrometer analyzing TOF. NMR spectra were recorded on Bruker AV-400 MHz in ppm (δ) downfield of TMS (6=0). Signal splitting patterns were described as singlet (s), doublet (d), triplet (t) or multiplet (m), with coupling constants (J) in hertz. Assignments were aided by COSY and HSQC experiments. High resolution mass spectra (HRMS) were performed by Analytical Instrument Center at the School of Pharmacy on an Electron Spray Injection (ESI) mass spectrometer.

General procedure A for the amide formation: To a solution of alkynoic acid (1.2 equiv.) in DMF was added DIPEA (3 equiv.) at 0° C. It was then followed by the addition of HATU (2 equiv.) After being stirred at room temperature for 30 min, a solution of amine (1 equiv.) in DMF was added. The reaction mixture was stirred for another 3 h at room temperature. The reaction mixture was then diluted with ethyl acetate and the organic phase was washed with water. The collected organic phase was dried over MgSO₄, after the filtration and concentration, the residue was submitted to the flash column chromatography for purification (Hex/EA: 1/1 to 1/2), giving the desired compound as a white foam.

General procedure B for the Sonogashira cross coupling: To a solution of alkyne (1 equiv.) and bromide (2 equiv.) in dry DMF was added copper iodide (0.2 equiv.) and Pd(PPh₃)Cl₂ (0.1 equiv.). The solution was purged and refilled with Argon 3 times. Triethylamine (same amount of DMF) was then added and the solution was purged again with Argon. The reaction mixture was stirred at 70° C. overnight. After being cooled down to room temperature, the solvent was removed under reduced pressure and the black tar residue was submitted to the flash column chromatography for purification (DCM/MeOH: 99/1-95/5, gradually), giving the desired compound as a light-yellow foam.

General procedure C for the N-alkylation: To a solution of amine (1 equiv.) in THF was added potassium carbonate (2 equiv.). It was followed by the addition of bromide or iodide (2 equiv.). The reaction mixture was refluxed overnight. The reaction was then quenched at room temperature. Water was added, and the aqueous phase was extracted with dichloromethane. The collected organic phase was dried over MgSO₄. After the filtration and concentration, the residue was submitted to the flash column chromatography for purification (DCM/MeOH: 99/1 to 98/2), giving the desired compound as a white foam.

### Tert-butyl 4-(2-(4-(tert-butyl)-2-ethoxyphenyl)-4,5-bis(4-chlorophenyl)-4,5-dihydro-1H-imidazole-1-carbonyl)piperazine-1-carboxylate (3a)

To a suspension of imidazole hydrochloride 3 (1.0 g, 1.98 mmol, 1 equiv.) in dry dichloromethane (20 mL) was added DIPEA (3.11 mL, 17.86 mmol, 9 equiv.) at 0° C. When the solution became clear, it was added to a solution of triphosgene (442 mg, 1.49 mmol, 0.75 equiv.) in dry dichloromethane (20 mL) at 0° C. The reaction mixture was stirred at room temperature for 3 h. A solution of N-Boc-piperazine (591 mg, 3.18 mmol, 1.6 equiv.) in dry dichloromethane (10 mL) was then added and the reaction mixture was stirred for another 2.5 h. The reaction was diluted with dichloromethane and washed with water. The collected organic phase was dried over MgSO₄. After the filtration and concentration, the residue was submitted to flash column chromatography for purification (DCM-DCM/Methanol 98/2), giving the desired compound 3a (1.0 g, 74%) as a white foam. ¹H NMR (400 MHz, CDCl₃): δ 7.54 (d, 1H, J=8.0 Hz, H—Ar), 7.12-6.85 (m, 10H, 10×H—Ar), 5.64 (d, 1H, J=9.8 Hz), 5.47 (d, 1H, J=9.8 Hz), 4.11 (dt, 2H, J=16.0, 8.8 Hz), 3.04 (bd, 4H), 2.81 (bd, 4H), 1.45 (t, 3H), 1.39 (s, 9H), 1.33 (s, 9H). ¹³C NMR (101 MHz, CDCl₃): δ 160.38, 156.94, 156.35, 155.73, 154.43, 136.32, 135.26, 133.02, 132.84, 130.39, 129.28 (2C), 128.44 (2C), 128.09 (2C), 127.93 (2C), 117.73, 117.26, 108.70, 80.19, 71.61, 69.18, 64.01, 45.70 (4C), 35.24, 31.21 (3C), 28.28 (3C), 14.97. HRMS (ESI): Calcd. For C₃₇H₄₄Cl₂N₄O₄ [M+H]¹⁺: 679.2812; Found: 679.2798 (−2.0 ppm).

### (2-(4-(tert-butyl)-2-ethoxyphenyl)-4,5-bis(4-chlorophenyl)-4,5-dihydro-1H-imidazol-1-yl)(piperazin-1-yl)methanone (1)

To a solution of 3a (1.0 g, 1.47 mmol, 1 equiv.) in dichloromethane (50 mL) was added trifluoroacetic acid (3.38 mL, 44.14 mmol, 30 equiv.). The reaction mixture was stirred at room temperature for 1 h. After the starting material was fully consumed, the reaction mixture was poured into saturated sodium bicarbonate and stirred for 30 min. The aqueous phase was extracted with dichloromethane. The collected organic phase was dried over MgSO₄, filtered and concentrated to give the desired compound 1 (0.647 g, 76%) as a white foam. ¹H NMR (400 MHz, CDCl₃): δ 7.52 (d, 1H, J=8.0 Hz, H—Ar), 7.12-7.00 (m, 5H, 5×H—Ar), 7.00-6.93 (m, 3H, 3×H—Ar), 6.87 (d, 2H, J=8.4 Hz, 2×H—Ar), 5.65 (d, 1H, J=9.9 Hz), 5.46 (d, 1H, J=9.9 Hz), 4.21-4.03 (m, 2H), 3.07 (t, 4H, J=4.8 Hz), 2.33 (dd, 4H, J=11.0, 6.2 Hz), 1.46 (t, 3H, J=7.0 Hz), 1.35 (s, 9H). ¹³C NMR (101 MHz, CDCl₃): δ 160.83, 156.96, 156.21, 155.67, 136.58, 135.49, 133.04, 132.86, 130.39, 129.44 (2C), 128.60 (2C), 128.16 (2C), 127.99 (2C), 117.73, 117.46, 108.79, 71.72, 69.29, 64.08, 46.99 (2C), 45.20 (2C), 35.34, 31.38 (3C), 14.96. HRMS (ESI): Calcd. For C₃₂H₃₇Cl₂N₄O₂ [M+H]¹⁺: 579.2288; Found: 579.2287 (−0.1 ppm).

### 1-(4-(2-(4-(tert-butyl)-2-ethoxyphenyl)-4,5-bis(4-chlorophenyl)-4,5-dihydro-1H-imidazole-1-carbonyl)piperazin-1-yl)pent-4-yn-1-one (4)

According to the procedure A, product 4 (yield: 98%) was obtained as a white foam. ¹H NMR (400 MHz, CDCl₃): δ 7.54 (d, 1H, J=8.0 Hz, H—Ar), 7.09-7.01 (m, 5H, 5×H—Ar), 6.99-6.88 (m, 3H, 3×H—Ar), 6.85 (d, 2H, J=8.2 Hz, 2×H—Ar), 5.64 (d, 1H, J=9.9 Hz), 5.48 (d, 1H J=9.8 Hz), 4.20-4.02 (m, 2H), 3.11-3.01 (m, 4H), 2.93-2.88 (m, 4H), 2.43 (m, 4H), 1.92 (s, 1H), 1.44 (t, 3H, J=6.9 Hz), 1.32 (s, 9H). ¹³C NMR (101 MHz, CDCl₃): δ 169.36, 160.52, 157.04, 156.78, 155.62, 136.12, 135.11, 133.29, 133.06, 130.51, 129.34 (2C), 128.57 (2C), 128.25 (2C), 128.09 (2C), 117.92, 117.03, 108.86, 83.21, 71.49, 69.31, 69.02, 64.25, 46.06, 45.86, 44.46, 40.82, 35.40, 32.08, 31.34 (3C), 14.94, 14.52. HRMS (ESI): Calcd. For C₃₇H₄OCl₂N₄O₃ [M+H]¹⁺: 659.2550; Found: 659.2546 (−0.6 ppm).

### 1-(4-(2-(4-(tert-butyl)-2-ethoxyphenyl)-4,5-bis(4-chlorophenyl)-4,5-dihydro-1H-imidazole-1-carbonyl)piperazin-1-yl)hex-5-yn-1-one (5)

According to the procedure A, product 5 (yield: 98%) was obtained as a white foam. ¹H NMR (400 MHz, CDCl₃): δ 7.54 (d, 1H, J=8.0 Hz, H—Ar), 7.12-7.05 (m, 3H, 3×H—Ar), 7.03 (d, 2H, J=8.4 Hz, 2×H—Ar), 6.98-6.91 (m, 3H, 3×H—Ar), 6.86 (d, 2H, J=8.3 Hz, 2×H—Ar), 5.64 (d, 1H, J=9.8 Hz), 5.48 (d, 1H J=9.8 Hz), 4.19-4.05 (m, 2H), 3.15-2.84 (m, 8H), 2.32 (t, 2H, J=7.3 Hz), 2.21 (td, 2H, J=6.7, 2.5 Hz), 1.93 (t, 1H, J=2.5 Hz), 1.82-1.71 (m, 2H), 1.45 (t, 3H, J=7.0 Hz), 1.33 (s, 9H). ¹³C NMR (101 MHz, CDCl₃): δ 170.71, 160.34, 157.05, 156.62, 155.78, 136.31, 135.28, 133.22, 133.01, 130.51, 129.38 (2C), 128.56 (2C), 128.24 (2C), 128.08 (2C), 117.88, 117.33, 108.86, 83.64, 71.74, 69.31, 69.26, 64.22, 46.14, 45.86, 44.50, 40.67, 35.38, 31.40, 31.34 (3C), 23.71, 17.93, 14.96. HRMS (ESI): Calcd. For C₃₇H₄OCl₂N₄O₃ [M+H]¹⁺: 673.2712; Found: 673.2696 (−2.4 ppm).

### 1-(4-(2-(4-(tert-butyl)-2-ethoxyphenyl)-4,5-bis(4-chlorophenyl)-4,5-dihydro-1H-imidazole-1-carbonyl)piperazin-1-yl)hept-6-yn-1-one (6)

According to the procedure A, product 6 (yield: 97%) was obtained as a white foam. ¹H NMR (400 MHz, CDCl₃): δ 7.55 (d, 1H, J=8.0 Hz, H—Ar), 7.13-6.91 (m, 8H, 8×H—Ar), 6.86 (d, 2H, J=8.3 Hz, 2×H—Ar), 5.64 (d, 1H, J=9.8 Hz), 5.48 (d, 1H J=9.8 Hz), 4.21-4.04 (m, 2H), 3.15-2.82 (m, 8H), 2.28-2.13 (m, 4H), 1.92 (t, 1H, J=2.5 Hz), 1.70-1.56 (m, 2H), 1.56-1.41 (m, 5H), 1.32 (s, 9H). ¹³C NMR (101 MHz, CDCl₃): δ 171.13, 160.35, 157.07, 156.63, 155.82, 136.32, 135.29, 133.24, 133.04, 130.52, 129.39 (2C), 128.57 (2C), 128.26 (2C), 128.10 (2C), 117.89, 117.36, 108.87, 84.06, 71.77, 69.32, 68.78, 64.24, 46.21, 45.88, 44.61, 40.65, 35.40, 32.66, 31.36 (3C), 28.05, 24.25, 18.27, 14.98. HRMS (ESI): Calcd. For C₃₉H₄₅Cl₂N₄O₃ [M+H]¹⁺: 687.2863; Found: 687.2842 (−3.0 ppm).

### 3-(4-(5-(4-(2-(4-(tert-butyl)-2-ethoxyphenyl)-4,5-bis(4-chlorophenyl)-4,5-dihydro-1H-imidazole-1-carbonyl)piperazin-1-yl)-5-oxopent-1-yn-1-yl)-1-oxoisoindolin-2-yl)piperidine-2,6-dione (7)

According to the procedure B, product 7 (yield: 31%) was obtained as a light-yellow foam. ¹H NMR (400 MHz, CDCl₃): δ 8.22 (d, J=10.7 Hz, 1H), 7.80 (d, J=7.4, Hz, 1H), 7.63-7.49 (m, 2H), 7.42 (d, J=7.5 Hz, 1H), 7.03 (m, 6H), 6.92 (d, J=8.0 Hz, 2H), 6.83 (t, J=7.5 Hz, 2H), 5.64 (d, J=9.4 Hz, 1H), 5.51 (br, 1H), 5.21 (td, J=12.9, 5.2 Hz, 1H), 4.45 (dd, J=16.7, 4.5 Hz, 1H), 4.28 (dd, J=16.7, 11.4 Hz, 1H), 4.19-4.05 (m, 2H), 3.16-2.78 (m, 10H), 2.71 (d, J=7.8 Hz, 2H), 2.50 (t, J=7.2 Hz, 2H), 2.33 (m, 1H), 2.22 (m, 1H), 1.46 (t, J=6.5 Hz, 3H), 1.30 (s, 9H). ¹³C NMR (101 MHz, CDCl₃): δ 171.05, 169.66, 169.63, 169.16, 169.03, 157.05, 143.73, 143.71, 135.78, 134.72, 134.65, 133.44, 133.31, 131.67, 130.34, 129.38 (2C), 128.71, 128.57 (2C), 128.27 (2C), 128.20 (2C), 123.69, 119.21, 117.97, 109.04, 108.97, 94.52, 77.48, 77.36, 77.16, 76.84, 71.69, 69.29, 64.40, 51.91, 46.98, 46.12, 45.89, 44.35, 35.44, 32.33, 31.67, 31.32 (3C), 23.65, 15.51, 15.00. HRMS (ESI): Calcd. For C₅₀H₅₁Cl₂N₆O₆ [M+H]¹⁺: 901.3242; Found: 901.3216 (−2.8 ppm).

### 3-(4-(6-(4-(2-(4-(tert-butyl)-2-ethoxyphenyl)-4,5-bis(4-chlorophenyl)-4,5-dihydro-1H-imidazole-1-carbonyl)piperazin-1-yl)-6-oxohex-1-yn-1-yl)-1-oxoisoindolin-2-yl)piperidine-2,6-dione (8)

According to the procedure B, product 8 (yield: 35%) was obtained as a light-yellow foam. ¹H NMR (400 MHz, CDCl₃): δ 8.54 (d, J=14.4 Hz, 1H), 7.81 (d, J=7.4 Hz, 1H), 7.62-7.51 (m, 2H), 7.44 (t, J=7.6 Hz, 1H), 7.15-6.77 (m, 10H), 5.63 (d, J=9.9 Hz, 1H), 5.51 (d, J=9.5 Hz, 1H), 5.22 (td, J=8.8, 4.5 Hz, 1H), 4.46-4.38 (m, 1H), 4.35-4.27 (m, 1H), 4.22-4.00 (m, 2H), 3.13-2.68 (m, 10H), 2.49 (t, J=6.8 Hz, 2H), 2.33 (m, 3H), 2.23-2.14 (m, 1H), 1.92-1.83 (m, 2H), 1.44 (t, J=6.7 Hz, 3H), 1.31 (s, 9H). ¹³C NMR (101 MHz, CDCl₃): δ 171.09, 170.97, 170.43, 169.75, 169.60, 168.94, 168.91, 156.95, 143.50, 134.55, 133.18, 133.07, 131.60, 130.45, 129.28 (2C), 128.58, 128.50, 128.45, 128.18, 128.07 (2C), 128.04 (2C), 123.51, 119.24, 117.83, 108.72, 94.91, 94.89, 71.54, 69.16, 64.20, 51.73, 46.79, 46.07, 45.69, 44.32, 40.57, 35.30, 31.54 (2C), 31.20 (3C), 23.83, 23.53, 18.99, 14.84. HRMS (ESI): Calcd. For C₅₁H₅₃Cl₂N₆O₆ [M+H]¹⁺: 915.3398; Found: 915.3400 (+0.2 ppm).

### 3-(4-(7-(4-(2-(4-(tert-butyl)-2-ethoxyphenyl)-4,5-bis(4-chlorophenyl)-4,5-dihydro-1H-imidazole-1-carbonyl)piperazin-1-yl)-7-oxohept-1-yn-1-yl)-1-oxoisoindolin-2-yl)piperidine-2,6-dione (9)

According to the procedure B, product 9 (yield: 19%) was obtained as a light-yellow foam. ¹H NMR (400 MHz, CDCl₃): δ 8.26 (s, 1H), 7.80 (d, J=7.6 Hz, 1H), 7.62-7.49 (m, 2H), 7.42 (t, J=7.7 Hz, 1H), 7.17-6.68 (m, 10H), 5.63 (t, J=9.4 Hz, 1H), 5.49 (d, J=10.0 Hz, 1H), 5.23 (dt, J=13.4, 5.1 Hz, 1H), 4.46 (d, J=16.8 Hz, 1H), 4.33 (dd, J=16.8, 5.2 Hz, 1H), 4.12 (dt, J=25.5, 8.0 Hz, 2H), 3.18-2.62 (m, 10H), 2.45 (m, 3H), 2.20 (m, 3H), 1.81-1.51 (m, 4H), 1.45 (t, J=7.1 Hz, 3H), 1.31 (s, 9H). ¹³C NMR (101 MHz, CDCl₃): δ 171.24, 171.00, 169.67, 169.15, 157.07, 156.76, 143.85, 134.53, 133.27, 133.12, 131.70, 130.47, 129.40 (2C), 128.65, 128.60, 128.51, 128.23 (2C), 128.14 (2C), 127.92, 123.46, 119.52, 117.92, 114.06, 108.86, 95.43, 77.36, 71.76, 69.30, 64.29, 51.90, 47.11, 46.16, 45.80, 44.54, 40.65, 35.40, 32.62, 31.71, 31.33 (3C), 28.23, 24.32, 23.46, 19.45, 14.97. HRMS (ESI): Calcd. For C₅₂H₅₅Cl₂N₆O₆ [M+H]¹⁺: 929.3555; Found: 929.3537 (−1.9 ppm).

### 2-amino-1-(4-(2-(4-(tert-butyl)-2-ethoxyphenyl)-4,5-bis(4-chlorophenyl)-4,5-dihydro-1H-imidazole-1-carbonyl)piperazin-1-yl)ethan-1-one (10)

According to the procedure A: the desired Boc protected glycine derivative (0.19 g) was obtained as a white foam. It was then dissolved in dichloromethane (5 mL) and trifluoroacetic acid (0.592 mL, 7.74 mmol, 30 equiv.) was added into the solution. The reaction mixture was stirred at room temperature for 1 h. After the starting material was fully consumed, the reaction mixture was poured into saturated sodium bicarbonate and stirred for 30 min. The aqueous phase was extracted with dichloromethane. The collected organic phase was dried over MgSO₄, filtered and concentrated to give the desired compound 10 (0.15 g, 91% in two steps) as a white foam. ¹H NMR (400 MHz, CDCl₃): δ 7.55 (d, J=8.0 Hz, 1H), 7.09 (dd, J=5.1, 3.3 Hz, 3H), 7.03 (d, J=8.5 Hz, 2H), 6.96 (dd, J=11.7, 4.8 Hz, 3H), 6.86 (d, J=8.3 Hz, 2H), 5.63 (d, J=9.8 Hz, 1H), 5.49 (d, J=9.8 Hz, 1H), 4.20-4.01 (m, 2H), 3.32 (br, 1H), 3.99-2.05 (m, 9H), 1.45 (t, J=7.0 Hz, 3H), 1.32 (s, 9H). ¹³C NMR (101 MHz, CDCl₃): δ 171.14, 160.27, 157.07, 156.69, 155.83, 136.30, 135.28, 133.26, 133.05, 130.57, 129.39 (2C), 128.57 (2C), 128.26 (2C), 128.11 (2C), 117.94, 117.35, 108.85, 77.48, 77.16, 76.84, 71.79, 69.35, 64.26, 46.10 (2C), 45.73 (2C), 43.29, 35.41, 31.37 (3C), 14.97. HRMS (ESI): Calcd. For C₃₄H₄₀Cl₂N₅O₃ [M+H]¹⁺: 636.2503; Found: 636.2493 (−1.5 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)pent-4-ynamide (11)

According to the procedure A, product 11 (yield: 83%) was obtained as a white foam. ¹H NMR (400 MHz, CDCl₃): δ 7.55 (d, J=8.0 Hz, 1H), 7.13-6.83 (m, 10H), 6.62 (br, 1H), 5.64 (d, J=9.8 Hz, 1H), 5.50 (d, J=9.8 Hz, 1H), 4.21-4.04 (m, 2H), 3.93 (d, J=3.8 Hz, 2H), 3.09 (m, 4H), 2.80 (br, 4H), 2.55-2.42 (m, 4H), 1.99 (t, J=2.4 Hz, 1H), 1.45 (t, J=6.8 Hz, 3H), 1.32 (s, 9H). ¹³C NMR (101 MHz, CDCl₃): δ 170.92, 166.31, 160.03, 156.95, 156.66, 155.62, 136.08, 135.06, 133.18, 132.96, 130.43, 129.24 (2C), 128.44 (2C), 128.15 (2C), 128.00 (2C), 117.84, 117.13, 108.75, 82.67, 71.60, 69.37, 69.20, 64.18, 45.67 (2C), 43.39 (2C), 41.17, 35.00, 31.23 (3C), 29.70, 14.85, 14.72. HRMS (ESI): Calcd. For C₃₉H₄₄Cl₂N₅O₄ [M+H]¹⁺: 716.2765; Found: 716.2760 (−0.6 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)hex-5-ynamide (12)

According to the procedure A, product 12 (yield: 89%) was obtained as a white foam. ¹H NMR (400 MHz, CDCl₃): δ 7.56 (d, J=8.0 Hz, 1H), 7.10 (d, J=8.3 Hz, 3H), 7.03 (d, J=8.4 Hz, 2H), 6.99-6.91 (m, 3H), 6.85 (d, J=8.3 Hz, 2H), 6.55 (s, 1H), 5.64 (d, J=9.8 Hz, 1H), 5.51 (d, J=9.8 Hz, 1H), 4.19-4.04 (m, 2H), 3.92 (d, J=3.9 Hz, 2H), 3.15-2.98 (m, 6H), 2.81 (m, 2H), 2.37 (t, J=7.4 Hz, 2H), 2.28-2.20 (m, 2H), 1.97 (d, J=2.5 Hz, 1H), 1.89-1.76 (m, 2H), 1.45 (t, J=12.7 Hz, 3H), 1.32 (s, 9H). ¹³C NMR (101 MHz, CDCl₃): δ 172.51, 166.61, 160.27, 157.07, 156.82, 155.68, 136.14, 135.11, 133.31, 133.09, 130.54, 129.35 (2C), 128.56 (2C), 128.27 (2C), 128.12 (2C), 117.95, 117.13, 108.88, 83.41, 71.56, 69.38, 69.30, 64.30, 45.78 (2C), 43.51, 41.17, 41.13, 35.41, 34.81, 31.34 (3C), 24.18, 17.98, 14.95. HRMS (ESI): Calcd. For C₄₀H₄₆Cl₂N₅O₄ [M+H]¹⁺: 730.2921; Found: 730.2917 (−0.6 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)hept-6-ynamide (13)

According to the procedure A, product 13 (yield: 89%) was obtained as a white foam. ¹H NMR (400 MHz, CDCl₃): δ 7.55 (d, J=8.0 Hz, 1H), 7.09 (d, J=6.7 Hz, 3H), 7.03 (d, J=8.4 Hz, 2H), 7.00-6.89 (m, 3H), 6.86 (d, J=8.3 Hz, 2H), 6.43 (s, 1H), 5.63 (d, J=9.8 Hz, 1H), 5.49 (d, J=9.8 Hz, 1H), 4.12 (dt, J=15.9, 8.8 Hz, 2H), 3.91 (d, J=3.8 Hz, 2H), 3.09 (m, 6H), 2.81 (m, 2H), 2.27-2.17 (m, 2H), 1.94 (t, J=2.6 Hz, 1H), 1.75 (m, 2H), 1.55 (m, 4H), 1.45 (t, J=12.8 Hz, 3H), 1.32 (s, 10H). ¹³C NMR (101 MHz, CDCl₃): δ 172.64, 166.46, 159.99, 156.95, 156.63, 155.65, 136.12, 135.10, 133.17, 132.94, 130.44, 129.25 (2C), 128.44 (2C), 128.14 (2C), 127.99 (2C), 117.83, 117.19, 108.75, 83.95, 71.65, 69.21, 68.65, 64.17, 45.68 (2C), 43.36, 41.05, 41.00, 35.78, 35.29, 31.23 (3C), 27.92, 24.64, 18.17, 14.85. HRMS (ESI): Calcd. For C₄₁H₄₈Cl₂N₅O₄ [M+H]¹⁺: 744.3078; Found: 744.3075 (−0.4 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)-5-(2-(2,6-dioxopiperidin-3-yl)-1-oxoisoindolin-4-yl)pent-4-ynamide (14)

According to the procedure B, product 14 (yield: 35%) was obtained as a light-yellow foam. ¹H NMR (400 MHz, CDCl₃): δ 8.23 (s, 1H), 7.80 (d, J=7.6 Hz, 1H), 7.55 (d, J=7.5 Hz, 1H), 7.45-7.35 (m, 2H), 7.03 (m, 5H), 6.97-6.80 (m, 5H), 6.66 (s, 1H), 5.67-5.49 (m, 2H), 5.21 (dd, J=13.2, 5.1 Hz, 1H), 4.49-4.39 (m, 1H), 4.32 (dd, J=16.8, 7.3 Hz, 1H), 4.13 (m, 2H), 3.94 (m, 2H), 3.06 (br, 6H), 2.90-2.75 (m, 6H), 2.54 (t, J=6.6 Hz, 2H), 2.45-2.34 (m, 1H), 2.19 (m, 1H), 1.46 (t, J=6.6 Hz, 3H), 1.27 (s, 9H). ¹³C NMR (101 MHz, CDCl₃): δ 171.14, 170.89, 169.67, 169.09, 166.46, 166.38, 157.06, 155.68, 155.40, 143.81, 134.89, 133.86, 133.71, 133.35, 131.67, 130.53, 130.11, 129.42 (2C), 128.65, 128.56, 128.53, 128.29 (2C), 128.25 (2C), 123.67, 119.18, 118.10, 114.07, 108.92, 100.13, 94.16, 71.65, 69.32, 64.42, 51.88, 47.11, 45.84, 45.79, 43.51, 41.30, 41.15, 35.44, 35.34, 31.67, 31.30 (3C), 23.53, 16.00, 14.99. HRMS (ESI): Calcd. For C₅₂H₅₄Cl₂N₇O₇ [M+H]¹⁺: 958.3456; Found: 958.3450 (−0.6 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)-6-(2-(2-methylene-6-oxopiperidin-3-yl)-1-oxoisoindolin-4-yl)hex-5-ynamide (15)

According to the procedure B, product 15 (yield: 39%) was obtained as a light-yellow foam. ¹H NMR (400 MHz, CDCl₃): δ 8.60 (s, 1H), 7.79 (d, J=7.6 Hz, 1H), 7.61-7.52 (m, 2H), 7.42 (t, J=7.6 Hz, 1H), 7.13-7.06 (m, 3H), 7.03 (d, J=8.3 Hz, 2H), 6.99-6.91 (m, 3H), 6.86 (d, J=7.9 Hz, 2H), 6.58 (s, 1H), 5.63 (d, J=9.8 Hz, 2H), 5.49 (d, J=9.8 Hz, 2H), 5.22 (dd, J=13.2, 5.1 Hz, 1H), 4.52 (dd, J=16.8, 4.9 Hz, 1H), 4.33 (d, J=16.7 Hz, 1H), 4.21-4.02 (m, 2H), 3.91 (m, 2H), 3.09 (br, 6H), 2.83 (m, 4H), 2.50 (t, J=6.7 Hz, 2H), 2.41 (t, J=7.5 Hz, 2H), 1.95 (m, 2H), 1.86-1.79 (m, 2H), 1.45 (t, J=7.0 Hz, 3H), 1.32 (s, 9H). ¹³C NMR (101 MHz, CDCl₃): δ 172.24, 171.34, 169.94, 169.11, 166.62, 160.14, 157.08, 156.82, 156.71, 155.86, 143.83, 136.22, 135.23, 134.58, 133.25, 133.05, 131.65, 130.58, 129.37 (2C), 128.57 (2C), 128.53, 128.24 (2C), 128.11 (2C), 123.47, 119.39, 117.94, 117.31, 108.86, 95.02, 95.00, 77.36, 71.74, 69.31, 64.29, 51.97, 51.94, 47.12, 45.80, 43.52, 41.17 (2C), 35.40, 34.82, 31.65, 31.35 (3C), 24.06, 23.53, 18.79, 14.96. HRMS (ESI): Calcd. For C₅₃H₅₆Cl₂N₇O₇ [M+H]¹⁺: 972.3613; Found: 972.3612 (−0.1 ppm).

### N-(2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)-7-(2-(2-methylene-6-oxopiperidin-3-yl)-1-oxoisoindolin-4-yl)hept-6-ynamide (16)

According to the procedure B, product 16 (yield: 24%) was obtained as a light-yellow foam. ¹H NMR (400 MHz, CDCl₃): δ 8.40 (s, 1H), 7.78 (d, J=7.5 Hz, 1H), 7.58-7.50 (m, 2H), 7.41 (t, J=7.6 Hz, 1H), 7.11-7.04 (m, 3H), 7.02 (d, J=8.5 Hz, 2H), 6.98-6.91 (m, 3H), 6.86 (d, J=8.3 Hz, 2H), 6.48 (t, J=3.8 Hz, 2H), 5.63 (d, J=9.8 Hz, 1H), 5.49 (d, J=9.8 Hz, 1H), 5.24 (dd, J=13.3, 5.1 Hz, 1H), 4.49 (d, J=16.9 Hz, 1H), 4.36 (d, J=16.9 Hz, 1H), 4.20-4.02 (m, 2H), 3.87 (t, J=3.5 Hz, 2H), 3.16-3.00 (m, 6H), 2.82 (m, 4H), 2.46 (m, 3H), 2.29-2.14 (m, 3H), 1.87-1.71 (m, 2H), 1.61 (m, 2H), 1.44 (t, J=6.9 Hz, 3H), 1.31 (s, 9H). ¹³C NMR (101 MHz, CDCl₃): δ 172.60, 171.53, 169.86, 169.18, 166.71, 160.20, 157.07, 156.72, 155.81, 144.05, 136.21, 135.22, 134.38, 133.25, 133.05, 131.66, 130.58, 129.38 (2C), 128.59 (2C), 128.48, 128.24 (2C), 128.1 (2C)1, 123.37, 119.58, 117.95, 117.26, 108.86, 95.62, 77.36, 71.73, 69.32, 64.29, 51.86, 47.16, 45.84, 41.14 (2C), 35.93, 35.40, 31.72, 31.35 (3C), 28.09, 24.77, 23.38, 19.33, 14.96. HRMS (ESI): Calcd. For C₅₄H₅₈Cl₂N₇O₇ [M+H]¹⁺: 986.3769; Found: 986.3759 (−1.0 ppm).

### 2-(4-(tert-butyl)-2-ethoxyphenyl)-4,5-bis(4-chlorophenyl)-4,5-dihydro-1H-imidazol-1-yl)(4-(prop-2-yn-1-yl)piperazin-1-yl)methanone (17)

According to the procedure C, product 17 (yield: 71%) was obtained as a white foam. ¹H NMR (400 MHz, CDCl₃): δ 7.53 (d, J=8.0 Hz, 1H), 7.13-6.92 (m, 8H), 6.90-6.82 (m, 2H), 5.64 (d, J=9.9 Hz, 1H), 5.47 (d, J=9.8 Hz, 1H), 4.19-4.01 (m, 2H), 3.15 (br, 4H), 3.04 (d, J=2.5 Hz, 1H), 2.20 (d, J=2.4 Hz, 1H), 2.06 (br, 4H), 1.45 (t, J=7.0 Hz, 3H), 1.35 (s, 9H). ¹³C NMR (101 MHz, CDCl₃): δ 160.76, 156.74, 156.10, 155.22, 136.37, 135.28, 132.96, 132.79, 130.31, 129.31 (2C), 128.50 (2C), 128.04 (2C), 127.90 (2C), 117.74, 117.21, 108.66, 78.34, 73.37, 71.54, 69.27, 63.97, 51.32, 46.78 (2C), 45.55 (2C), 35.23, 31.32 (3C), 14.84. HRMS (ESI): Calcd. For C₃₅H₃₉Cl₂N₄O₂ [M+H]¹⁺: 617.2444; Found: 617.2447 (+0.5 ppm).

### (4-(but-3-yn-1-yl)piperazin-1-yl)(2-(4-(tert-butyl)-2-ethoxyphenyl)-4,5-bis(4-chlorophenyl)-4,5-dihydro-1H-imidazol-1-yl)methanone (18)

According to the procedure C, product 18 (yield: 66%) was obtained as a white foam. ¹H NMR (400 MHz, CDCl₃): δ 7.52 (d, J=8.0 Hz, 1H), 7.11-7.04 (m, 3H), 7.02 (d, J=8.0 Hz, 2H), 6.99-6.92 (m, 3H), 6.87 (d, J=8.0 Hz, 2H), 5.64 (d, J=9.8 Hz, 1H), 5.45 (d, J=9.8 Hz, 1H), 4.20-4.03 (m, 2H), 3.13 (br, 4H), 2.39-2.29 (m, 2H), 2.26-2.20 (m, 2H), 1.98-1.82 (m, 5H), 1.46 (t, J=7.1 Hz, 3H), 1.36 (s, 9H). ¹³C NMR (101 MHz, CDCl₃): δ 160.67, 156.89, 156.07, 155.44, 136.42, 135.36, 132.93, 132.76, 130.34, 129.32 (2C), 128.47 (2C), 128.02 (2C), 127.88 (2C), 117.72, 117.30, 108.73, 82.25, 71.58, 69.26, 69.23, 63.97, 56.68, 51.82 (2C), 45.70 (2C), 35.23, 31.35 (3C), 16.80, 14.85. HRMS (ESI): Calcd. For C₃₆H₄₁Cl₂N₄O₂ [M+H]¹⁺: 631.2601; Found: 631.2594 (−1.1 ppm).

### 2-(4-(tert-butyl)-2-ethoxyphenyl)-4,5-bis(4-chlorophenyl)-4,5-dihydro-1H-imidazol-1-yl)(4-(pent-4-yn-1-yl)piperazin-1-yl)methanone (19)

According to the procedure C, product 19 (yield: 83%) was obtained as a white foam. ¹H NMR (400 MHz, CDCl₃): δ 7.54 (d, 1H, J=8.0 Hz, H—Ar), 7.09-7.01 (m, 5H, 5×H—Ar), 6.99-6.88 (m, 3H, 3×H—Ar), 6.85 (d, 2H, J=8.2 Hz, 2×H—Ar), 5.64 (d, 1H, J=9.9 Hz), 5.48 (d, 1H J=9.8 Hz), 4.20-4.02 (m, 2H), 3.11-3.01 (m, 4H), 2.93-2.88 (m, 4H), 2.43 (m, 4H), 1.92 (s, 1H), 1.44 (t, 3H, J=6.9 Hz,), 1.32 (s, 9H). ¹³C NMR (101 MHz, CDCl₃): δ 160.69, 156.89, 156.01, 155.46, 136.44, 135.39, 132.90, 132.74, 130.32, 129.33 (2C), 128.46 (2C), 128.02 (2C), 127.87 (2C), 117.70, 117.33, 108.75, 83.86, 71.60, 69.26, 68.54, 63.95, 56.96, 52.07 (2C), 45.80 (2C), 35.22, 31.36 (3C), 25.55, 16.21, 14.85. HRMS (ESI): Calcd. For C₃₇H₄₃Cl₂N₄O₂ [M+H]¹⁺: 645.2758; Found: 675.2749 (−1.4 ppm).

### 3-(4-(3-(4-(2-(4-(tert-butyl)-2-ethoxyphenyl)-4,5-bis(4-chlorophenyl)-4,5-dihydro-1H-imidazole-1-carbonyl)piperazin-1-yl)prop-1-yn-1-yl)-1-oxoisoindolin-2-yl)piperidine-2,6-dione (20)

To a solution of secondary amine (18 mg, 0.031 mmol, 1 equiv.) in acetone (5 mL) was added potassium carbonate (9 mg, 0.062 mmol, 2 equiv.). It was followed by the addition of chloride 24 (11 mg, 0.034 mmol, 1.1 equiv.) and sodium iodide (5 mg, 0.031 mmol, 1 equiv.). The reaction mixture was refluxed for 4 h. Then reaction was cooled down to room temperature and water was added into the solution. The organic phase was extracted by dichloromethane. The collected organic phase was dried over MgSO₄, after the filtration and concentration, the residue was submitted to flash column chromatography for purification (DCM/MeOH: 98/2 to 97/3), giving the desired compound 20 (0.018 g, 68%) as a light-yellow foam. ¹H NMR (400 MHz, CDCl₃): δ 7.98-7.77 (m, 2H), 7.55 (m, 2H), 7.46 (t, J=7.6 Hz, 1H), 7.07 (m, 3H), 6.96 (m, 5H), 6.87 (m, 2H), 5.66 (t, 1H), 5.46 (t, 1H), 5.22-5.15 (m, 1H), 4.44 (dd, J=16.7, 10.7 Hz, 1H), 4.35-4.01 (m, 3H), 3.30 (d, J=16.7 Hz, 2H), 3.16 (br, 4H), 2.96-2.72 (m, 2H), 2.35 (m, 1H), 2.18 (m, 5H), 1.45 (t, J=7.0 Hz, 3H), 1.35 (s, 9H). ¹³C NMR (101 MHz, CDCl₃): δ 171.18, 171.15, 169.60, 168.76, 160.94, 156.74, 156.27, 143.57, 136.15, 135.15, 134.90, 132.98, 132.84, 131.71, 130.35, 129.28 (2C), 129.25, 128.52, 128.45, 128.39, 127.97 (2C), 127.92 (2C), 124.08, 118.19, 117.75, 113.91, 108.64, 89.30, 81.24, 71.53, 69.14, 64.04, 51.76, 51.60, 51.51, 47.67, 47.01, 45.56, 35.25, 31.50, 31.31 (3C), 23.35, 14.82. HRMS (ESI): Calcd. For C₄₈H₄₉Cl₂N₆O₅ [M+H]¹⁺: 859.3136; Found: 859.3132 (−0.5 ppm).

### 3-(4-(4-(4-(2-(4-(tert-butyl)-2-ethoxyphenyl)-4,5-bis(4-chlorophenyl)-4,5-dihydro-1H-imidazole-1-carbonyl)piperazin-1-yl)but-1-yn-1-yl)-1-oxoisoindolin-2-yl)piperidine-2,6-dione (21)

According to the procedure B, product 21 (yield: 22%) was obtained as a light-yellow foam. ¹H NMR (400 MHz, CDCl₃): δ 8.13 (s, 1H), 7.80 (d, J=7.4 Hz, 1H), 7.57-7.49 (m, 2H), 7.43 (t, J=7.6 Hz, 1H), 7.12-6.92 (m, 8H), 6.88 (d, J=8.3 Hz, 2H), 5.65 (dd, J=12.5, 9.7 Hz, 1H), 5.51-5.41 (m, 1H), 5.22 (ddd, J=13.4, 8.9, 5.3 Hz, 1H), 4.48 (dd, J=16.7, 10.4 Hz, 1H), 4.26 (dd, J=16.7, 7.9 Hz, 1H), 4.20-4.00 (m, 2H), 3.14 (br, 4H), 2.94-2.73 (m, 1H), 2.54-2.16 (m, 7H), 1.95 (br, 4H), 1.44 (t, J=7.0 Hz, 3H), 1.35 (s, 9H). ¹³C NMR (101 MHz, CDCl₃): δ 170.95, 169.52, 169.45, 168.93, 168.90, 156.93, 156.27, 143.64, 136.12, 134.61, 134.48, 133.04, 132.93, 131.54, 129.29 (2C), 128.60, 128.50, 128.43, 128.05 (2C), 127.96 (2C), 123.52, 119.17, 117.75, 108.80, 93.53, 71.46, 69.19, 64.06, 56.67, 53.81, 51.79, 51.72, 46.87, 45.82, 35.27, 34.68, 31.60, 31.50, 31.46, 31.36 (3C), 22.66, 17.88, 14.86. HRMS (ESI): Calcd. For C₄₉H₅₂Cl₂N₆O₅ [M+H+H]²⁺: 437.1682; Found: 437.1681 (−0.4 ppm).

### 3-(4-(5-(4-(2-(4-(tert-butyl)-2-ethoxyphenyl)-4,5-bis(4-chlorophenyl)-4,5-dihydro-1H-imidazole-]-carbonyl)piperazin-1-yl)pent-1-yn-1-yl)-1-oxoisoindolin-2-yl)piperidine-2,6-dione (22)

According to the procedure B, product 22 (yield: 11%) was obtained as a light-yellow foam. ¹H NMR (400 MHz, CDCl₃): δ 7.81 (d, J=7.5 Hz, 1H), 7.53 (d, J=7.7 Hz, 2H), 7.44 (d, J=7.6 Hz, 1H), 7.12-6.93 (m, 8H), 6.86 (d, J=8.0 Hz, 2H), 5.66 (d, 1H), 5.46 (d, J=9.9 Hz, 1H), 5.24 (dt, J=13.3, 4.7 Hz, 1H), 4.48 (dd, J=16.6, 3.0 Hz, 1H), 4.29 (dd, J=16.6, 2.4 Hz, 1H), 4.13 (m, 1H), 3.13 (br, 4H), 2.97-2.75 (m, 2H), 2.46-2.18 (m, 6H), 2.04 (br, 2H), 1.91 (br, 2H), 1.68 (m, 2H), 1.47 (t, J=6.9 Hz, 3H), 1.34 (s, 9H). HRMS (ESI): Calcd. For C₅₀H₅₃Cl₂N₆O₅ [M+H]¹⁺: 887.3449; Found: 887.3413 (−4.0 ppm).

### 3-(4-(3-hydroxyprop-1-yn-1-yl)-1-oxoisoindolin-2-yl)piperidine-2, 6-dione (23)

According to the procedure B, alcohol 23 was obtained as a yellow solid with a yield of 81%. ¹H NMR (400 MHz, DMSO-d6): δ 11.01 (s, 1H), 7.76 (d, J=8.0 Hz, 1H), 7.69 (d, J=7.7 Hz, 1H), 7.56 (t, J=7.6 Hz, 1H), 5.76 (s, 1H), 5.43 (t, J=6.0 Hz, 1H), 5.16 (dd, J=5.2, 13.3 Hz, 1H), 4.48 (d, J=17.8 Hz, 1H), 4.37-4.32 (m, 3H), 2.92 (ddd, J=17.8, 13.6, 5.3 Hz, 1H), 2.68-2.41 (m, 2H), 2.04-2.00 (m, 1H). ¹³C NMR (101 MHz, DMSO-d6): δ 173.34, 171.47, 168.03, 144.35, 134.60, 132.50, 129.19, 123.66, 118.48, 95.65, 79.84, 52.07, 49.95, 46.24, 31.66, 22.87. HRMS (ESI): Calcd. For C₁₆H₁₅N₂O₄ [M+H]¹⁺: 299.1026; Found: 299.1023 (−1.0 ppm).

### 3-(4-(3-chloroprop-1-yn-1-yl)-1-oxoisoindolin-2-yl)piperidine-2,6-dione (24)

To a solution of alcohol 23 (0.62 g, 2.08 mmol, 1 equiv.) in dichloromethane (25 mL) was added thionyl chloride (0.181 mL, 2.49 mmol, 1.2 equiv.). It was followed by the addition of triethylamine (0.435 mL, 3.12 mmol, 1.5 equiv.). The reaction was stirred at room temperature for 1 h. The water was then added, and the aqueous phase was extracted with dichloromethane. The collected organic phase was dried over magnesium sulfate and filtered. The filtrate was evaporated and dried under vacuum, giving product 24 (0.42 g, 64%) without further purification.

### 4-(4-(2-(4-(tert-butyl)-2-ethoxyphenyl)-4,5-bis(4-chlorophenyl)-4,5-dihydro-1H-imidazole-1-carbonyl)piperazin-1-yl)-2-(2,6-dioxopiperidin-3-yl)isoindoline-1,3-dione (25)

To a solution of nutlin derivative 1 (45 mg, 0.078 mmol, 1 equiv.) in DMF (2 mL) was added DIPEA (0.027 mL, 0.156 mmol, 2 equiv.). It was followed by the addition of 2-(2,6-dioxopiperidin-3-yl)-4-fluoroisoindoline-1,3-dione (24 mg, 0.086 mmol, 1.1 equiv.). The reaction mixture was stirred at 90° C. overnight. The solvent was evaporated, and the residue was submitted to flash column chromatography for purification (DCM/MeOH: 99/1 to 98/2), giving product 25 (18 mg, 27%) as a light-yellow foam. ¹H NMR (400 MHz, CDCl₃): δ 8.21 (s, 1H), 7.60-7.51 (m, 2H), 7.40 (d, J=7.2 Hz, 1H), 7.13-7.00 (m, 5H), 6.98-6.85 (m, 6H), 5.67 (d, J=9.8 Hz, 1H), 5.47 (d, J=9.8 Hz, 1H), 4.92 (dd, J=12.1, 5.1 Hz, 1H), 4.21-4.05 (m, 2H), 3.42-3.25 (m, 4H), 3.00-2.46 (m, 7H), 2.13-2.04 (m, 1H), 1.46 (t, J=6.9 Hz, 3H), 1.26 (s, 9H). ¹³C NMR (101 MHz, CDCl₃): δ 170.93, 168.27, 167.19, 166.56, 160.64, 157.22, 156.42, 155.79, 149.92, 136.40, 135.79, 135.34, 134.19, 133.13, 132.98, 130.61, 129.41 (2C), 128.59 (2C), 128.22 (2C), 128.09 (2C), 123.20, 118.35, 117.86, 117.49, 116.64, 108.89, 71.67, 69.36, 69.32, 64.23, 50.20 (2C), 49.30, 45.99, 35.34, 31.39 (3C), 31.06, 22.75, 14.94. HRMS (ESI): Calcd. For C₄₅H₄₅Cl₂N₆O₆ [M+H]¹⁺: 835.2772; Found: 835.2768 (−0.5 ppm).

### 4-((2-(4-(2-(4-(tert-butyl)-2-ethoxyphenyl)-4,5-bis(4-chlorophenyl)-4,5-dihydro-1H-imidazole-1-carbonyl)piperazin-1-yl)-2-oxoethyl)amino)-2-(2,6-dioxopiperidin-3-yl)isoindoline-1,3-dione (26)

To a solution of compound 10 (50 mg, 0.079 mmol, 1 equiv.) in DMF (2 mL) was added DIPEA (0.027 mL, 0.157 mmol, 2 equiv.). It was followed by the addition of 2-(2,6-dioxopiperidin-3-yl)-4-fluoroisoindoline-1,3-dione (24 mg, 0.086 mmol, 1.1 equiv.). The reaction mixture was stirred at 90° C. for overnight. The solvent was evaporated, and the residue was submitted to flash column chromatography for purification (DCM/MeOH: 99/1 to 98/2), giving product 26 (23 mg, 33%) as a light-yellow foam. ¹H NMR (400 MHz, CDCl₃): δ 8.14 (s, 1H), 7.57 (d, J=8.0 Hz, 1H), 7.46 (d, J=7.9 Hz, 1H), 7.15-7.08 (m, 4H), 7.03 (d, J=8.4 Hz, 2H), 6.96 (m, 3H), 6.89-6.84 (m, 2H), 6.70 (d, J=8.5 Hz, 1H), 5.64 (d, J=9.8 Hz, 1H), 5.50 (d, J=9.8 Hz, 1H), 4.91 (dd, J=12.2, 5.3 Hz, 1H), 4.20-4.03 (m, 2H), 3.89 (d, J=4.4 Hz, 2H), 3.19-2.68 (m, 11H), 2.14-2.05 (m, 1H), 1.45 (t, J=7.0 Hz, 3H), 1.32 (s, 9H). ¹³C NMR (101 MHz, CDCl₃) δ 171.08, 168.99, 168.34, 167.63, 165.98, 160.19, 157.10, 156.79, 155.88, 145.45, 136.24, 136.22, 135.24, 133.30, 133.09, 132.81, 130.60, 129.38 (2C), 128.58 (2C), 128.28 (2C), 128.14 (2C), 117.96, 117.33, 116.86, 112.36, 111.38, 108.89, 71.80, 69.37, 64.31, 49.08, 46.21, 45.59, 44.10, 43.53, 41.29, 35.42, 31.54, 31.37 (3C), 22.84, 14.98. HRMS (ESI): Calcd. For C₄₇H₄₈Cl₂N₇O₇ [M+H]¹⁺: 892.2987; Found: 892.2974 (−1.4 ppm).

### Tert-butyl ((1R,2S)-2-(4-(tert-butyl)-2-ethoxybenzamido)-1,2-bis(4-chlorophenyl)ethyl) carbamate (28)

To a solution of diamine 27 (0.184 g, 0.482 mmol, 1 equiv.) and benzoic acid (0.118 g, 0.530 mmol, 1.1 equiv.) in dry DCM (10 mL), was added EDC (0.12 g, 0.627 mmol, 1.3 equiv.) at 0° C. It was followed by the addition of DMAP (0.006 g, 0.048 mmol, 0.1 equiv.). The reaction mixture was stirred at room temperature for 16 h. TLC (Hex/EA 3/1) showed that all starting material has been consumed. Water was then added, and the aqueous phase was extracted by DCM. The collected organic phase was dried over MgSO₄. After filtration and concentration, the residue was submitted to flash column chromatography for purification (Hex/EA: 5/1 to 3/1), giving compound 28 (0.27 g, 96%) as a white foam. ¹H NMR (400 MHz, CDCl₃): δ 8.51 (br, 1H), 8.15 (d, 1H, J=8.3 Hz), 7.30-7.18 (m, 4H), 7.12 (d, 1H, J=8.3), 7.03-6.89 (m, 5H), 5.87-5.72 (m, 2H), 5.08 (s, 1H), 4.23-4.11 (m, 2H), 1.36 (m, 18H), 1.26 (s, 3H). ¹³C NMR (101 MHz, CDCl₃): δ 165.65, 157.54, 156.85, 155.07, 136.71, 136.68, 133.65, 133.48, 132.28, 128.75 (2C), 128.59 (2C), 128.50 (2C), 128.28 (2C), 118.56, 117.78, 109.29, 80.00, 64.51, 59.71, 56.64, 35.26, 31.11 (3C), 29.71, 28.33 (3C), 14.64. HRMS (ESI): Calcd. For C₃₂H₃₈Cl₂N₄O₄Na [M+Na]¹⁺: 607.2101; Found: 607.2096 (−0.8 ppm).

### N-((1S,2R)-2-amino-1,2-bis(4-chlorophenyl)ethyl)-4-(tert-butyl)-2-ethoxybenzamide (28a)

To a solution of Boc protected amine-amide 28 (0.184 g, 0.314 mmol, 1 equiv.) in dichloromethane (10 mL) was added trifluoroacetic acid (0.722 mL, 9.43 mmol, 30 equiv.). The reaction mixture was stirred at room temperature for 3 h. After the starting material was fully consumed, the reaction mixture was poured into saturated sodium bicarbonate solution and stirred for 30 min. The aqueous phase was extracted with dichloromethane. The collected organic phase was dried over MgSO₄, filtered and concentrated to give compound 28a (0.14 g, 92%) as a white foam. ¹H NMR (400 MHz, CDCl₃): δ 8.92 (d, 1H, J=7.9 Hz), 8.10-8.06 (d, 1H, J=8.0 Hz), 7.27-7.18 (m, 4H), 7.10-7.05 (d, 1H, J=8.3 Hz), 7.06-6.95 (m, 5H), 5.43 (dd, 1H, J=8.1, 4.8 Hz), 4.40 (dd, 1H, J=11.9, 6.7 Hz), 4.24 (q, 2H, J=7.0 Hz), 1.52 (t, 3H, J=7.0 Hz), 1.43 (d, 2H, J=7.0 Hz), 1.32 (s, 9H). ¹³C NMR (101 MHz, CDCl₃): δ 164.73, 156.94, 156.87, 140.64, 136.83, 133.25, 133.18, 132.09, 129.13 (2C), 128.36 (4C), 128.21 (2C), 118.40, 109.26, 77.34, 77.22, 77.02, 76.70, 64.49, 59.00, 58.66, 35.19, 31.13 (3C), 15.02. HRMS (ESI): Calcd. For C₂₇H₃₁Cl₂N₂O₂ [M+H]¹⁺: 485.1757; Found: 485.1753 (−0.9 ppm).

### Tert-butyl 4-(((1R,2S)-2-(4-(tert-butyl)-2-ethoxybenzamido)-1,2-bis(4-chlorophenyl)ethyl)carbamoyl)piperazine-1-carboxylate (29)

To a solution of amine-amide 28a (0.175 g, 0.360 mmol, 1 equiv.) in dry dichloromethane (10 mL) was added 1,1′-Carbonyldiimidazole (0.07 mg, 0.432 mmol, 1.2 equiv.). The reaction mixture was stirred at room temperature for 3 h. N-Boc-Piperazine (0.134 mg, 0.721 mmol, 2 equiv.) was added and the reaction mixture was stirred at room temperature overnight. Water was then added, and the mixture was extracted with DCM. The collected organic phase was dried over MgSO₄, after the filtration and concentration, the residue was submitted to flash column chromatography for purification (Hex/EA: 3/1 to 2/1), giving compound 29 (0.21 g, 84%) as a white foam. ¹H NMR (400 MHz, CDCl₃): δ 8.52 (d, 1H, J=8.1 Hz,), 8.21 (d, 1H, J=8.3 Hz), 7.51 (d, 1H, J=4.7 Hz), 7.30-7.25 (m, 2H), 7.16 (m, 3H), 6.96 (m, 3H), 6.86 (d, 2H, J=8.4 Hz), 5.79 (dd, 1H, J=8.1, 2.1 Hz), 5.09 (dd, 1H, J=4.5, 2.3 Hz), 4.12 (m, 2H), 3.41 (m, 8H), 1.47 (s, 9H), 1.34 (s, 9H), 1.14 (t, 3H, J=7.0 Hz). ¹³C NMR (101 MHz, CDCl₃): δ 167.35, 158.23, 157.17, 156.98, 154.82, 136.76, 136.60, 134.01, 133.45, 132.31, 129.56 (2C), 128.81 (2C), 128.49 (2C), 128.06 (2C), 118.80, 117.28, 109.54, 80.16, 64.77, 62.31, 57.73, 43.58 (4C), 35.46, 31.22 (3C), 28.55 (3C), 14.67. HRMS (ESI): Calcd. For C₃₇H₄₆Cl₂N₄O₅ [M+H]¹⁺: 697.2918; Found: 697.2911 (−0.9 ppm).

### ((4S,5R)-2-(4-(tert-butyl)-2-ethoxyphenyl)-4,5-bis(4-chlorophenyl)-4,5-dihydro-1H-imidazol-1-yl)(piperazin-1-yl)methanone ((4S, 5R)-1)

To a solution of Ph₃PO (0.415 g, 1.49 mmol, 4 equiv.) in dry dichloromethane (20 mL) was added triflic anhydride (0.125 mL, 0.745 mmol, 2 equiv.) dropwise at 0° C. The reaction mixture was stirred at 0° C. for 20 min. It was then followed by the addition of a solution of amide-urea 29 (0.26 g, 0.372 mmol, 1 equiv.) in DCM (5 mL). The reaction mixture was stirred at 0° C. for another 1.5 h. Water was then added, and the mixture was extracted with DCM. The collected organic phase was dried over MgSO₄, after the filtration and concentration, the residue was submitted to flash column chromatography for purification (DCM/methanol: 9/1), giving compound (4S, 5R)-1 (0.21 g, 97%) as a white foam. ¹H NMR (400 MHz, CDCl₃): δ 7.52 (d, 1H, J=8.0 Hz, H—Ar), 7.12-7.00 (m, 5H, 5×H—Ar), 7.00-6.93 (m, 3H, 3×H—Ar), 6.87 (d, 2H, J=8.4 Hz, 2×H—Ar), 5.65 (d, 1H, J=9.9 Hz), 5.46 (d, 1H J=9.9 Hz), 4.21-4.03 (m, 2H), 3.07 (t, 4H, J=4.8 Hz,), 2.33 (dd, 4H, J=11.0, 6.2 Hz,), 1.46 (t, 3H, J=7.0 Hz), 1.35 (s, 9H). ¹³C NMR (101 MHz, CDCl₃): δ 160.83, 156.96, 156.21, 155.67, 136.58, 135.49, 133.04, 132.86, 130.39, 129.44 (2C), 128.60 (2C), 128.16 (2C), 127.99 (2C), 117.73, 117.46, 108.79, 71.72, 69.29, 64.08, 46.99 (2C), 45.20 (2C), 35.34, 31.38 (3C), 14.96.

### (4-(but-3-yn-1-yl)piperazin-1-yl)((4S,5R)-2-(4-(tert-butyl)-2-ethoxyphenyl)-4,5-bis(4-chlorophenyl)-4,5-dihydro-1H-imidazol-1-yl)methanone (30)

According to the procedure C, product 30 (yield: 61%) was obtained as a white foam.

### ((4S,5R)-2-(4-(tert-butyl)-2-ethoxyphenyl)-4,5-bis(4-chlorophenyl)-4,5-dihydro-1H-imidazol-1-yl)(4-(pent-4-yn-1-yl)piperazin-1-yl)methanone (31)

According to the procedure C, product 31 (yield: 72%) was obtained as a white foam.

### 3-(4-(3-(4-((4S,5R)-2-(4-(tert-butyl)-2-ethoxyphenyl)-4,5-bis(4-chlorophenyl)-4,5-dihydro-1H-imidazole-1-carbonyl)piperazin-1-yl)prop-1-yn-1-yl)-1-oxoisoindolin-2-yl)piperidine-2,6-dione (32)

To a solution of (4S, 5R)-1 (400 mg, 0.69 mmol, 1 equiv.) in acetone (50 mL) was added potassium carbonate (190 mg, 1.38 mmol, 2 equiv.). It was then followed by the addition of chloride 24 (240 mg, 0.076 mmol, 1.1 equiv.) and sodium iodide (103 mg, 0.69 mmol, 1 equiv.). The reaction mixture was refluxed for 4 h. The reaction was then cooled down to room temperature and quenched by water. The organic phase was extracted by dichloromethane. The collected organic phase was dried over MgSO₄. After the filtration and concentration, the residue was submitted to flash column chromatography for purification (DCM/MeOH: 98/2 to 97/3), giving compound 32 (0.35 g, 59%) as a light-yellow foam.

### 3-(4-(4-(4-((4S,5R)-2-(4-(tert-butyl)-2-ethoxyphenyl)-4,5-bis(4-chlorophenyl)-4,5-dihydro-1H-imidazole-1-carbonyl)piperazin-1-yl)but-1-yn-1-yl)-1-oxoisoindolin-2-yl)piperidine-2,6-dione (33)

According to the procedure B, product 33 (yield: 28%) was obtained as a light-yellow foam.

### 3-(4-(5-(4-((4S,5R)-2-(4-(tert-butyl)-2-ethoxyphenyl)-4,5-bis(4-chlorophenyl)-4,5-dihydro-1H-imidazole-1-carbonyl)piperazin-1-yl)pent-1-yn-1-yl)-1-oxoisoindolin-2-yl)piperidine-2,6-dione (34)

According to the procedure B, product 34 (yield: 10%) was obtained as a light-yellow foam.

### Biological Evaluation:

Cell Culture:

The human acute leukemia RS4;11 cell line was purchased from American Type Culture Collection (ATCC Accession No. CRL-1873), and cultured in RPMI-1640 media (Corning Incorporated (USA), One Riverfront Plaza, Corning, N.Y. 14831) supplemented with 10% fetal bovine serum (FBS), 1% sodium pyruvate, 1% penicillin/streptomycin, and 10 mM HEPES at 37° C. under a humidified 5% CO₂ atmosphere. The experiments were performed by using cells with 8-20 passages after purchasing.

Cell Viability Assay:

Cells were seeded in a 96-well cell culture plate at a density of 2×10⁴ cells with 90 μL cell culture media overnight. The cells were then treated with the indicated dose of compounds, respectively, and incubated at 37° C. under a humidified 5% CO₂ atmosphere for 48 h. After the incubation, the cells were treated with 10 μL of MTT solution (5 mg/mL) and incubated with another 4 h, followed by the addition of 100 μL of solubilizing solution. The plate was placed on the shaker for overnight at room temperature until the formazan were entirely dissolved. The plate was scanned at 570 nm and 690 nm. The absorbance was normalized to the DMSO-treated cells, and the IC₅₀ was calculated by non-linear regression analysis using GraphPad Prism 6 software.

Western Blot Assay:

Cells were treated with the indicated concentrations of compounds under the indicated time. Cells were collected and lysed with RIPA buffer (Corning) after being washed with cold PBS twice. The supernatant was collected after centrifugation at 16,000×g at 4° C. for 15 min. The protein concentration was measured with BCA (bicinchoninic acid) method. (Smith, P. K., et al. (1985) “Measurement of protein using bicinchoninic acid,” Anal. Biochem. 150(1):76-85.) The protein expressed in the whole cell lysates was identified by Western Blot. A quantity of 40 μg protein per sample was loaded and then separated by SDS-PAGE. Proteins in the gel was transferred to polyvinylidene fluoride (PVDF) membranes, then blocked with 5% non-fat milk, and probed with the primary antibodies at 4° C. overnight. The primary antibodies are as following: MDM2 (D1V2Z) (1:1000), p53 (DO-7) (1:1000), PARP (46D11) (1:1000), Caspase-3 (1:1000), and p21 Waf1/Cip 1(12D1) (1:1000), all of which were purchased from Cell Signaling Technology, Inc., 3 Trask Lane, Danvers, Mass. 01923, along with a-Tubulin (1:1000) and β-Actin (1:1000), which were purchased from R&D Systems, Inc., 614 McKinley Place NE, Minneapolis, Minn. 55413. The membrane was washed with TBS-T 3 times, and incubated with HRP-linked secondary antibodies for 1 h at room temperature, followed by washing with TBS-T. The membrane was incubated with ECL (Bio-rad) for 5 min. The protein band was visualized by using a Bio-Rad Chemi-Doc MP imaging system. The intensity of the band was measured by Image J software.

qRT-PCR Analysis:

Total RNA was extracted using GeneJET RNA Purification Kit (Thermo Scientific) and titrated by SYNERGY H1 Hybrid Multi-Mode Reader (BioTek) at 260 nm. 1 μg of total RNA was subjected to reverse transcription using High-Capacity cDNA Reverse Transcription Kit (appliedbiosystems). Quantitative RT-PCR was performed using PowerUp™ SYBR™ Green Master Mix (appliedbiosystems) and the amplification detected in a QuantStudio 7 Flex Real-Time PCR System (ThermoFisher Applied Biosystems). Primer sequence:

MDM2 Forward:
(SEQ. ID. NO: 1)
5′-GGCAGGGGAGAGTGATACAGA-3′
Reverse:
(SEQ. ID. NO: 2)
5′-GAAGCCAATTCTCACGAAGGG-3′
TP53 Forward:
(SEQ. ID. NO: 3)
5′-GAGCTGAATGAGGCCTTGGA-3′
Reverse:
(SEQ. ID. NO: 4)
5′-CTGAGTCAGGCCCTTCTGTCTT-3′
CDKN1A Forward:
(SEQ. ID. NO: 5)
5′-AGGTGGACCTGGAGACTCTCAG-3′
Reverse:
(SEQ. ID. NO: 6)
5′-TCCTCTTGGAGAAGATCAGCCG-3′
GAPDH Forward:
(SEQ. ID. NO: 7)
5′-CTCCTCTGACTTCAACAGCGACAC-3′
Reverse:
(SEQ. ID. NO: 8)
5′-TGCTGTAGCCAAATTCGTTGTCAT-3′

Flow Cytometry:

Cells were seeded in a 6 cm circular cell culture dish at a density of 5×10⁶ cells. After being settled overnight, the cells were treated with the indicated concentrations of compounds for 24 h. The collected cells were double stained with FITC Annexin V and PI according to the manufacturer's protocols. The apoptosis was analyzed using a FITC Annexin V apoptosis detection kit (556547, BD Biosciences, 2350 Qume Drive, San Jose, Calif. 95131).

### REFERENCES

[1] B. Vogelstein, D. Lane, A. J. Levine, Surfing the p53 network, Nature, 408 (2000) 307-310.
[2] A. J. Levine, p53, the cellular gatekeeper for growth and division, Cell, 88 (1997) 323-331.
[3] A. Feki, I. Irminger-Finger, Mutational spectrum of p53 mutations in primary breast and ovarian tumors, Crit Rev Oncol Hemat, 52 (2004) 103-116.
[4] D. A. Freedman, L. Wu, A. J. Levine, Functions of the MDM2 oncoprotein, Cell Mol Life Sci, 55 (1999) 96-107.
[5] S. M. Picksley, D. P. Lane, The p53-mdm2 autoregulatory feedback loop: a paradigm for the regulation of growth control by p53?, Bioessays, 15 (1993) 689-690.
[6] P. H. Kussie, S. Gorina, V. Marechal, B. Elenbaas, J. Moreau, A. J. Levine, N. P. Pavletich, Structure of the MDM2 oncoprotein bound to the p53 tumor suppressor transactivation domain, Science, 274 (1996) 948-953.
[7] C. J. Brown, S. Lain, C. S. Verma, A. R. Fersht, D. P. Lane, Awakening guardian angels: drugging the p53 pathway, Nat Rev Cancer, 9 (2009) 862-873.
[8] M. Wade, Y. C. Li, G. M. Wahl, MDM2, MDMX and p53 in oncogenesis and cancer therapy, Nat Rev Cancer, 13 (2013) 83-96.
[9] I. R. Hardcastle, Inhibitors of the MDM2-p53 interaction as anticancer drugs, Drug Future, 32 (2007) 883-896.
[10] L. T. Vassilev, MDM2 inhibitors for cancer therapy, Trends Mol Med, 13 (2007) 23-31.
[11] B. Zhang, B. T. Golding, I. R. Hardcastle, Small-molecule MDM2-p53 inhibitors: recent advances, Future Med Chem, 7 (2015) 631-645.
[12] Y. J. Zhao, A. Aguilar, D. Bernard, S. M. Wang, Small-Molecule Inhibitors of the MDM2-p53 Protein-Protein Interaction (MDM2 Inhibitors) in Clinical Trials for Cancer Treatment, J Med Chem, 58 (2015) 1038-1052.
[13] N. Atatreh, M. A. Ghattas, S. K. Bardaweel, S. Al Rawashdeh, M. Al Sorkhy, Identification of new inhibitors of Mdm2-p53 interaction via pharmacophore and structure-based virtual screening, Drug Des Dev Ther, 12 (2018) 3741-3752.
[14] P. Brandao, J. B. Loureiro, S. Carvalho, M. H. Hamadou, S. Cravo, J. Moreira, D. Pereira, A. Palmeira, M. Pinto, L. Saraiva, H. Cidade, Targeting the MDM2-p53 protein-protein interaction with prenylchalcones: Synthesis of a small library and evaluation of potential antitumor activity, European Journal of Medicinal Chemistry, 156 (2018) 711-721.
[15] G. C. Liao, D. Y. Yang, L. L. Ma, W. W. Li, L. Q. Hu, L. M. Zeng, P. Wu, L. X. Duan, Z. Q. Liu, The development of piperidinones as potent MDM2-P53 protein-protein interaction inhibitors for cancer therapy, European Journal of Medicinal Chemistry, 159 (2018) 1-9.
[16] H. J. Yi, X. L. Yan, Q. Y. Luo, L. P. Yuan, B. X. Li, W. T. Pan, L. Zhang, H. B. Chen, J. Wang, Y. B. Zhang, Y. F. Zhai, M. Z. Qiu, D. J. Yang, A novel small molecule inhibitor of MDM2-p53 (APG-115) enhances radiosensitivity of gastric adenocarcinoma, J Exp Clin Canc Res, 37 (2018) 97 doi.org/10.1186/s13046-13018-10765-13048.
[17] M. J. Hansen, F. M. Feringa, P. Kobauri, W. Szymanski, R. H. Medema, B. L. Feringa, Photoactivation of MDM2 Inhibitors: Controlling Protein-Protein Interaction with Light, J Am Chem Soc, 140 (2018) 13136-13141.
[18] M. Gicquel, C. Gomez, M. C. G. Alvarez, O. Pamlard, V. Guerineau, E. Jacquet, J. Bignon, A. Voituriez, A. Marinetti, Inhibition of p53-Murine Double Minute 2 (MDM2) Interactions with 3,3′-Spirocyclopentene Oxindole Derivatives, J Med Chem, 61 (2018) 9386-9392.
[19] G. M. Burslem, P. Ottis, S. Jaime-Figueroa, A. Morgan, P. M. Cromm, M. Toure, C. M. Crews, Efficient Synthesis of Immunomodulatory Drug Analogues Enables Exploration of Structure-Degradation Relationships, Chemmedchem, 13 (2018) 1508-1512.
[20] B. Vu, P. Wovkulich, G. Pizzolato, A. Lovey, Q. Ding, N. Jiang, J. J. Liu, C. Zhao, K. Glenn, Y. Wen, C. Tovar, K. Packman, L. Vassilev, B. Graves, Discovery of RG7112: A Small-Molecule MDM2 Inhibitor in Clinical Development, ACS Med Chem Lett, 4 (2013) 466-469.
[21] C. Iancu-Rubin, M. Goar, R. Hoffman, Disruption of MDM2-p53 Interactions by a Small Molecule Inhibitor RG7112 Increases Apoptosis and Impairs Polyploidization of Human Megakaryocytes, Blood, 120 (2012) 1082.
[22] M. Zanjirband, R. J. Edmondson, J. Lunec, Pre-clinical efficacy and synergistic potential of the MDM2-p53 antagonists, Nutlin-3 and RG7388, as single agents and in combined treatment with cisplatin in ovarian cancer, Oncotarget, 7 (2016) 40115-40134.
[23] L. D. Chen, R. F. Rousseau, S. A. Middleton, G. L. Nichols, D. R. Newell, J. Lunec, D. A. Tweddle, Pre-clinical evaluation of the MDM2-p53 antagonist RG7388 alone and in combination with chemotherapy in neuroblastoma, Oncotarget, 6 (2015) 10207-10221.
[24] D. Q. Sun, Z. H. Li, Y. Rew, M. Gribble, M. D. Bartberger, H. P. Beck, J. Canon, A. Chen, X. Q. Chen, D. Chow, J. Deignan, J. Duquette, J. Eksterowicz, B. Fisher, B. M. Fox, J. S. Fu, A. Z. Gonzalez, F. G. L. De Turiso, J. B. Houze, X. Huang, M. Jiang, L. X. Jin, F. Kayser, J. W. Liu, M. C. Lo, A. M. Long, B. Lucas, L. R. McGee, J. McIntosh, J. Mihalic, J. D. Oliner, T. Osgood, M. L. Peterson, P. Roveto, A. Y. Saiki, P. Shaffer, M. Toteva, Y. C. Wang, Y. C. Wang, S. Wortman, P. Yakowec, X. L. Yan, Q. P. Ye, D. Y. Yu, M. Yu, X. N. Zhao, J. Zhou, J. Zhu, S. H. Olson, J. C. Medina, Discovery of AMG 232, a Potent, Selective, and Orally Bioavailable MDM2-p53 Inhibitor in Clinical Development, J Med Chem, 57 (2014) 1454-1472.
[25] S. M. Wang, W. Sun, Y. J. Zhao, D. McEachern, I. Meaux, C. Barriere, J. A. Stuckey, J. L. Meagher, L. C. Bai, L. Liu, C. G. Hoffman-Luca, J. F. Lu, S. Shangary, S. H. Yu, D. Bernard, A. Aguilar, O. Dos-Santos, L. Besret, S. Guerif, P. Pannier, D. Gorge-Bernat, L. Debussche, SAR405838: An Optimized Inhibitor of MDM2-p53 Interaction That Induces Complete and Durable Tumor Regression, Cancer Res, 74 (2014) 5855-5865.
[26] C. Holohan, S. Van Schaeybroeck, D. B. Longley, P. G. Johnston, Cancer drug resistance: an evolving paradigm, Nature Reviews Cancer, 13 (2013) 714-726.
[27] K. M. Sakamoto, K. B. Kim, A. Kumagai, F. Mercurio, C. M. Crews, R. J. Deshaies, Protacs: Chimeric molecules that target proteins to the Skp1-Cullin-F box complex for ubiquitination and degradation, P Natl Acad Sci USA, 98 (2001) 8554-8559.
[28] C. Maniaci, S. J. Hughes, A. Testa, W. Z. Chen, D. J. Lamont, S. Rocha, D. R. Alessi, R. Romeo, A. Ciulli, Homo-PROTACs: bivalent small-molecule dimerizers of the VHL E3 ubiquitin ligase to induce self-degradation, Nature Communications, 8 (2017) 830, DOI: 810.1038/s41467-41017-00954-41461.
[29] V. Zoppi, S. J. Hughes, C. Maniaci, A. Testa, T. Gmaschitz, C. Wieshofer, M. Koegl, K. M. Riching, D. L. Daniels, A. Spallarossa, A. Ciulli, Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7, J Med Chem, 62 (2018) 699-726.
[30] A. Testa, X. Lucas, G. V. Castro, K. H. Chan, J. E. Wright, A. C. Runcie, M. S. Gadd, W. T. A. Harrison, E. J. Ko, D. Fletcher, A. Ciulli, 3-Fluoro-4-hydroxyprolines: Synthesis, Conformational Analysis, and Stereoselective Recognition by the VHL E3 Ubiquitin Ligase for Targeted Protein Degradation, J Am Chem Soc, 140 (2018) 9299-9313.
[31] J. Lu, Y. M. Qian, M. Altieri, H. Q. Dong, J. Wang, K. Raina, J. Hines, J. D. Winkler, A. P. Crew, K. Coleman, C. M. Crews, Hijacking the E3 Ubiquitin Ligase Cereblon to Efficiently Target BRD4, Chemistry & Biology, 22 (2015) 755-763.
[32] G. E. Winter, D. L. Buckley, J. Paulk, J. M. Roberts, A. Souza, S. Dhe-Paganon, J. E. Bradner, Phthalimide conjugation as a strategy for in vivo target protein degradation, Science, 348 (2015) 1376-1381.
[33] J. Lohbeck, A. K. Miller, Practical synthesis of a phthalimide-based Cereblon ligand to enable PROTAC development, Bioorg Med Chem Lett, 26 (2016) 5260-5262.
[34] Y. Demizu, N. Shibata, T. Hattori, N. Ohoka, H. Motoi, T. Misawa, T. Shoda, M. Naito, M. Kurihara, Development of BCR-ABL degradation inducers via the conjugation of an imatinib derivative and a cIAP1 ligand, Bioorg Med Chem Lett, 26 (2016) 4865-4869.
[35] S. Tomoshige, Y. Hashimoto, M. Ishikawa, Efficient protein knockdown of HaloTag-fused proteins using hybrid molecules consisting of IAP antagonist and HaloTag ligand, Bioorgan Med Chem, 24 (2016) 3144-3148.
[36] M. Naito, N. Ohoka, N. Shibata, SNIPERs-Hijacking IAP activity to induce protein degradation, Drug Discovery Today: Technologies, (2019) doi.org/10.1016/j.ddtec.2018.1012.1002.
[37] A. C. Lai, M. Toure, D. Hellerschmied, J. Salami, S. Jaime-Figueroa, E. Ko, J. Hines, C. M. Crews, Modular PROTAC Design for the Degradation of Oncogenic BCR-ABL, Angew Chem Int Edit, 55 (2016) 807-810.
[38] D. Remillard, D. L. Buckley, J. Paulk, G. L. Brien, M. Sonnett, H. S. Seo, S. Dastjerdi, M. Wuhr, S. Dhe-Paganon, S. A. Armstrong, J. E. Bradner, Degradation of the BAF Complex Factor BRD9 by Heterobifunctional Ligands, Angew Chem Int Edit, 56 (2017) 5738-5743.
[39] B. Zhou, J. T. Hu, F. M. Xu, Z. Chen, L. C. Bai, E. Fernandez-Salas, M. Lin, L. Liu, C. Y. Yang, Y. J. Zhao, D. McEachern, S. Przybranowski, B. Wen, D. X. Sun, S. M. Wang, Discovery of a Small-Molecule Degrader of Bromodomain and Extra-Terminal (BET) Proteins with Picomolar Cellular Potencies and Capable of Achieving Tumor Regression, J Med Chem, 61 (2018) 462-481.
[40] J. Hines, S. Lartigue, H. Dong, Y. Qian, C. M. Crews, MDM2-recruiting PROTAC Offers Superior, Synergistic Anti-proliferative Activity via Simultaneous Degradation of BRD4 and Stabilization of p53, Cancer Res, 79 (2018) 251-262.
[41] D. T. Saenz, W. Fiskus, Y. Qian, T. Manshouri, K. Rajapakshe, K. Raina, K. G. Coleman, A. P. Crew, A. Shen, C. P. Mill, B. Sun, P. Qiu, T. M. Kadia, N. Pemmaraju, C. DiNardo, M. S. Kim, A. J. Nowak, C. Coarfa, C. M. Crews, S. Verstovsek, K. N. Bhalla, Novel BET protein proteolysis-targeting chimera exerts superior lethal activity than bromodomain inhibitor (BETi) against post-myeloproliferative neoplasm secondary (s) AML cells, Leukemia, 31 (2017) 1951-1961.
[42] K. Raina, J. Lu, Y. M. Qian, M. Altieri, D. Gordon, A. M. K. Rossi, J. Wang, X. Chen, H. Q. Dong, K. Siu, J. D. Winkler, A. P. Crew, C. M. Crews, K. G. Coleman, PROTAC-induced BET protein degradation as a therapy for castration-resistant prostate cancer, P Natl Acad Sci USA, 113 (2016) 7124-7129.
[43] K. M. DeMars, C. J. Yang, C. I. Castro-Rivera, E. Candelario-Jalil, Selective degradation of BET proteins with dBET1, a proteolysis-targeting chimera, potently reduces pro-inflammatory responses in lipopolysaccharide-activated microglia, Biochem Bioph Res Co, 497 (2018) 410-415.
[44] K. Cyrus, M. Wehenkel, E. Y. Choi, H. Lee, H. Swanson, K. B. Kim, Jostling for Position: Optimizing Linker Location in the Design of Estrogen Receptor-Targeting PROTACs, Chemmedchem, 5 (2010) 979-985.
[45] K. Yang, Y. L. Song, H. B. Xie, H. Wu, Y. T. Wu, E. D. Leisten, W. P. Tang, Development of the first small molecule histone deacetylase 6 (HDAC6) degraders, Bioorg Med Chem Lett, 28 (2018) 2493-2497.
[46] C. M. Robb, J. I. Contreras, S. Kour, M. A. Taylor, M. Abid, Y. A. Sonawane, M. Zahid, D. J. Murry, A. Natarajan, S. Rana, Chemically induced degradation of CDK9 by a proteolysis targeting chimera (PROTAC), Chem Commun, 53 (2017) 7577-7580.
[47] J. M. Hatcher, E. S. Wang, L. Johannessen, N. Kwiatkowski, T. Sim, N. S. Gray, Development of Highly Potent and Selective Steroidal Inhibitors and Degraders of CDK8, Acs Med Chem Lett, 9 (2018) 540-545.
[48] J. L. Bian, J. Ren, Y. R. Li, J. B. Wang, X. Xu, Y. F. Feng, H. Tang, Y. J. Wang, Z. Y. Li, Discovery of Wogonin-based PROTACs against CDK9 and capable of achieving antitumor activity, Bioorg Chem, 81 (2018) 373-381.
[49] Y. Li, J. Yang, A. Aguilar, D. McEachern, S. Przybranowski, L. Liu, C. Y. Yang, M. Wang, X. Han, S. Wang, Discovery of MD-224 as a First-in-Class, Highly Potent, and Efficacious Proteolysis Targeting Chimera Murine Double Minute 2 Degrader Capable of Achieving Complete and Durable Tumor Regression, J Med Chem, 62 (2019) 448-466.
[50] L. T. Vassilev, B. T. Vu, B. Graves, D. Carvajal, F. Podlaski, Z. Filipovic, N. Kong, U. Kammlott, C. Lukacs, C. Klein, N. Fotouhi, E. A. Liu, In vivo activation of the p53 pathway by small-molecule antagonists of MDM2, Science, 303 (2004) 844-848.
[51] D. P. Bondeson, A. Mares, I. E. D. Smith, E. Ko, S. Campos, A. H. Miah, K. E. Mulholland, N. Routly, D. L. Buckley, J. L. Gustafson, N. Zinn, P. Grandi, S. Shimamura, G. Bergamini, M. Faelth-Savitski, M. Bantscheff, C. Cox, D. A. Gordon, R. R. Willard, J. J. Flanagan, L. N. Casillas, B. J. Votta, W. den Besten, K. Famm, L. Kruidenier, P. S. Carter, J. D. Harling, I. Churcher, C. M. Crews, Catalytic in vivo protein knockdown by small-molecule PROTACs, Nat Chem Biol, 11 (2015) 611-617.
[52] Y. Li, M. E. Stockton, I. Bhuiyan, B. E. Eisinger, Y. Gao, J. L. Miller, A. Bhattacharyya, X. Y. Zhao, MDM2 inhibition rescues neurogenic and cognitive deficits in a mouse model of fragile X syndrome, Sci Transl Med, 8 (2016) 336ra361, DOI: 310.1126/scitranslmed.aad9370.
[53] J. T. Liu, T. J. Do, C. J. Simmons, J. C. Lynch, W. Gu, Z. X. Ma, W. Xu, W. P. Tang, Total synthesis of diptoindonesin G and its analogues as selective modulators of estrogen receptors, Org Biomol Chem, 14 (2016) 8927-8930.
[54] T. Ito, H. Ando, T. Suzuki, T. Ogura, K. Hotta, Y. Imamura, Y. Yamaguchi, H. Handa, Identification of a Primary Target of Thalidomide Teratogenicity, Science, 327 (2010) 1345-1350.
[55] M. E. Matyskiela, S. Couto, X. D. Zheng, G. Lu, J. L. Hui, K. Stamp, C. Drew, Y. Ren, M. Wang, A. Carpenter, C. W. Lee, T. Clayton, W. Fang, C. C. Lu, M. Riley, P. Abdubek, K. Blease, J. Hartke, G. Kumar, R. Vessey, M. Rolfe, L. G. Hamann, P. P. Chamberlain, SALL4 mediates teratogenicity as a thalidomide-dependent cereblon substrate, Nat Chem Biol, 14 (2018) 981-987.
[56] L. H. Shu, P. Wang, W. Liu, C. Gu, A Practical Synthesis of a cis-4,5-Bis(4-chlorophenyl)imidazoline Intermediate for Nutlin Analogues, Org Process Res Dev, 16 (2012) 1866-1869.
[57] M. V. Proskurnina, N. A. Lozinskaya, S. E. Tkachenko, N. S. Zefirov, Reaction of aromatic aldehydes with ammonium acetate, Russ J Org Chem, 38 (2002) 1149-1153.
[58] T. A. Davis, A. E. Vilgelm, A. Richmond, J. N. Johnston, Preparation of (−)-Nutlin-3 Using Enantioselective Organocatalysis at Decagram Scale, J Org Chem, 78 (2013) 10605-10616.
[59] T. A. Davis, J. N. Johnston, Catalytic, enantioselective synthesis of stilbene cis-diamines: A concise preparation of (−)-Nutlin-3, a potent p53/MDM2 inhibitor, Chem Sci, 2 (2011) 1076-1079.
[60] A. C. Lai, C. M. Crews, Induced protein degradation: an emerging drug discovery paradigm, Nat Rev Drug Discov, 16 (2017) 101-114.
[61] G. M. Burslem, B. E. Smith, A. C. Lai, S. Jaime-Figueroa, D. C. McQuaid, D. P. Bondeson, M. Toure, H. Q. Dong, Y. M. Qian, J. Wang, A. P. Crew, J. Hines, C. M. Crews, The Advantages of Targeted Protein Degradation Over Inhibition: An RTK Case Study, Cell Chem Biol, 25 (2018) 67-77.

What is claimed is:

1. A compound selected from the group consisting of: 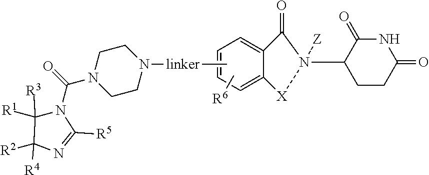
wherein each R¹, R², R³, R⁴, R⁵, and R⁶ are independently hydrogen or a substituent selected from the group consisting of: hydrogen, alkyl, alkenyl, alkynyl, alkoxy, halo, haloalkyl, hydroxy, hydroxyalkyl, aryl, alkyl-substituted aryl, alkoxy-substituted aryl, halo-substituted aryl, aroyl, (aryl)alkyl, heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl(alkyl)amine, and cyano;
dashed lines attached to X and Z are single bonds or are absent; when the dashed line attached to X is a single bond, X is —(C═O)— or —CH₂— and Z and the dashed line attached to Z are absent; when the dashed line attached to X is absent, X is hydrogen, Z is a hydrogen, and the dashed line attached to Z is a single bond;
“linker” is selected from the group consisting of a single bond, a C₁-C₁₂-alkylene, akenylene, or akynylene, —(C═O)—C₁-C₁₂-alkyl-, —(C═O)—C₁-C₁₂-alkenyl-, —(C═O)—C₁-C₁₂-alkynyl-, —(C═O)—C₁-C₁₂-alkylamino-, —(C═O)—C₁-C₁₂-alkenylamino-, —(C═O)—C₁-C₁₂-alkynylamino-, —(C═O)—C₁-C₁₂-alkylamido-, —(C═O)—C₁-C₁₂-alkenylamido-, —(C═O)—C₁-C₁₂-alkynylamido-, —(C═O)—C₁-C₁₂-alkylamido-C₁-C₁₂-alkyl/alkenyl/alkynyl-, —(C═O)—C₁-C₁₂-alkenylamido-C₁-C₁₂-alkyl/alkenyl/alkynyl-, —(C═O)—C₁-C₁₂-alkynylamido-C₁-C₁₂-alkyl/alkenyl/alkynyl-;
and salts thereof.

2. The compound of claim 1, wherein R⁵ is selected from the group consisting of: hydrogen, alkyl, alkoxy, halo, haloalkyl, hydroxy, hydroxyalkyl, aryl, alkyl-substituted aryl, alkoxy-substituted aryl, and halo-substituted aryl.

3. The compound of claim 2, wherein R⁶ is selected from the group consisting of hydrogen, alkyl, alkoxy, halo, haloalkyl, hydroxy, hydroxyalkyl, aryl, alkyl-substituted aryl, alkoxy-substituted aryl, and halo-substituted aryl.

4. The compound of claim 3, wherein R⁶ is selected from the group consisting of hydrogen, alkyl, alkoxy, halo, haloalkyl, hydroxy, and hydroxyalkyl.

5. The compound of claim 4, wherein R⁶ is selected from the group consisting of hydrogen, halo, and alkyl.

6. The compound of claim 1, wherein R⁵ is selected from the group consisting of hydrogen, alkyl, alkoxy, halo, haloalkyl, hydroxy, and hydroxyalkyl.

7. The compound of claim 1, wherein R⁵ is selected from the group consisting of aryl, alkyl-substituted aryl, alkoxy-substituted aryl, and halo-substituted aryl.

8. The compound of claim 1, wherein:
the dashed line attached to X is a single bond;
X is —(C═O)—;
Z is absent; and
the dashed line attached to Z is absent.

9. The compound of claim 1, wherein:
the dashed line attached to X is a single bond;
X is —(CH₂)—;
Z is absent; and
the dashed line attached to Z is absent.

10. The compound of claim 1, wherein:
the dashed line attached to X;
X is hydrogen;
the dashed line attached to Z is a single bond; and
Z is hydrogen.

11. The compound of claim 1, wherein one of R¹ or R³ is selected from the group consisting of aryl, alkyl-substituted aryl, alkoxy-substituted aryl, and halo-substituted aryl.

12. The compound of claim 1, wherein one of R² or R⁴ is selected from the group consisting of aryl, alkyl-substituted aryl, alkoxy-substituted aryl, and halo-substituted aryl.

13. The compound of claim 1, wherein one of R¹ or R³ and one of R² or R⁴ is selected from the group consisting of aryl, alkyl-substituted aryl, alkoxy-substituted aryl, and halo-substituted aryl.

14. The compound of claim 1, selected from the group consisting of: 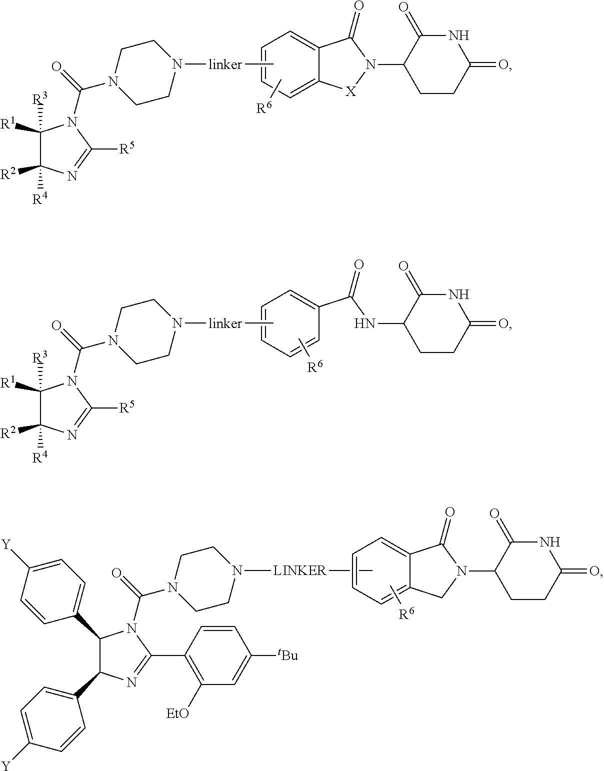
wherein R¹ and R² are not hydrogen, and R³ and R⁴ are hydrogen; and each Y is independently selected from the group consisting of hydrogen, halogen, or C₁-C₆-alkyl.

15. The compound of claim 14, selected from the group consisting of:

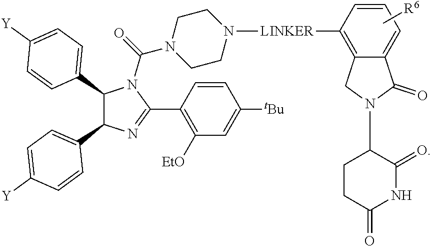

16. The compound of claim 14, wherein each Y is chlorine.

17. The compound of claim 1, which is selected from: 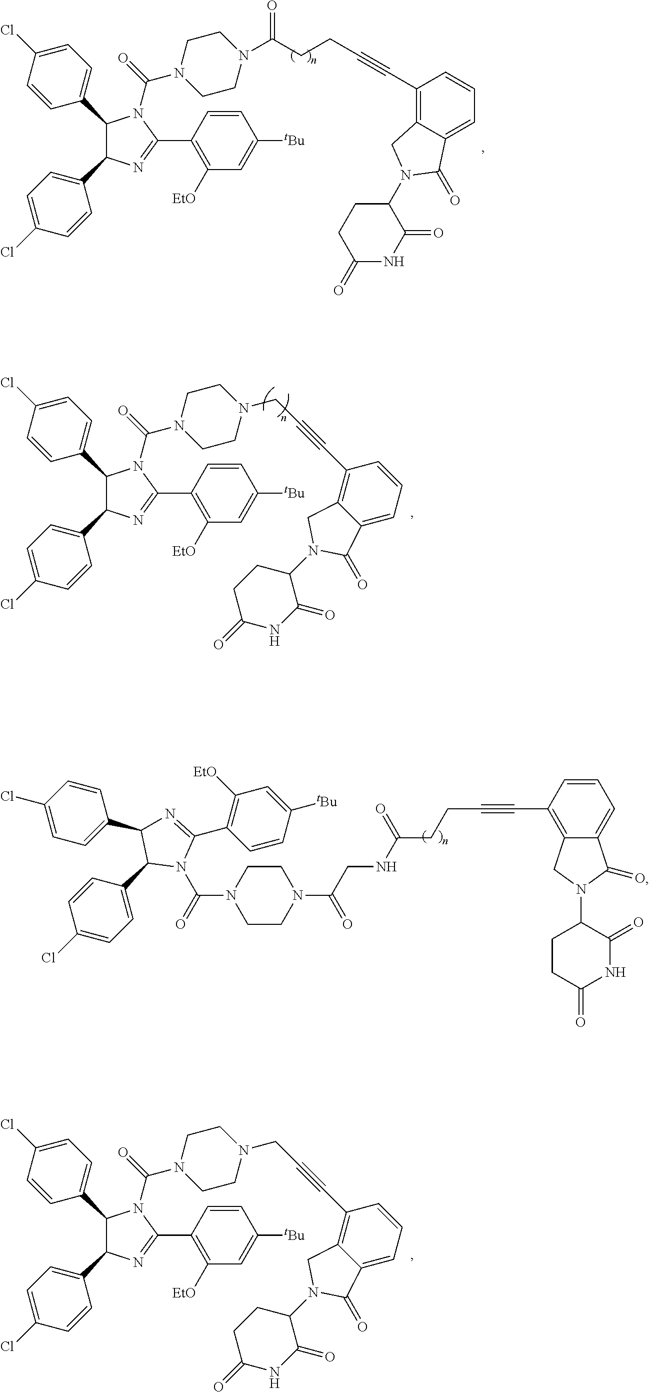 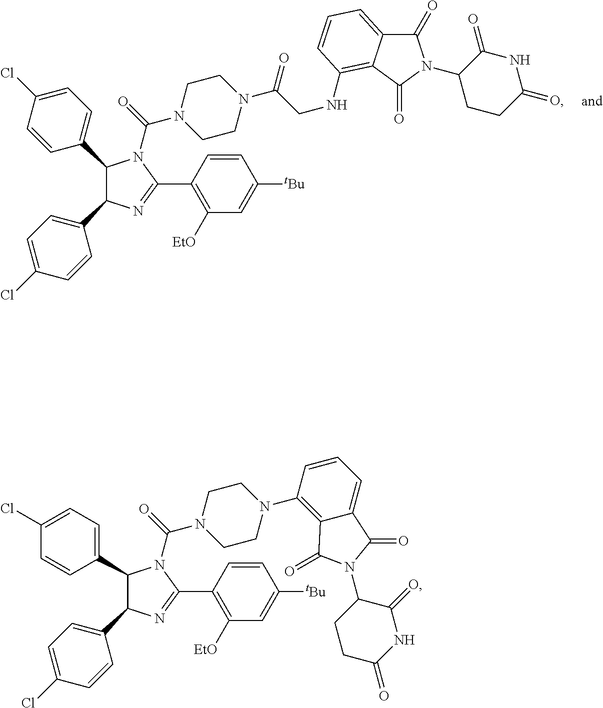 [SMILES: CCOc1cc(C(C)(C)C)ccc1C1=N[C@@H](c2ccc(Cl)cc2)[C@@H](c2ccc(Cl)cc2)N1C(=O)N1CCN(C(=O)CNc2cccc3c2C(=O)N(C2CCC(=O)NC2=O)C3=O)CC1 ; CCOc1cc(C(C)(C)C)ccc1C1=N[C@@H](c2ccc(Cl)cc2)[C@@H](c2ccc(Cl)cc2)N1C(=O)N1CCN(c2cccc3c2C(=O)N(C2CCC(=O)NC2=O)C3=O)CC1]
wherein “n” is an integer of from 1 to 3.

18. A pharmaceutical composition comprising an amount of one or more compounds as recited in claim 1, in combination with a pharmaceutically suitable carrier.

19. A method to inhibit neoplastic cell growth, the method comprising contacting a cell with a neoplastic cell growth-inhibiting amount of one or more compounds as recited claim 1.

20. The method of claim 19, the method comprising administering to a subject a neoplastic cell growth inhibiting-effective amount of one or more compounds as recited in claim 1.

21. The method of claim 20, comprising administering the one or more compounds of claim 1 to a mammalian subject.

22. The method of claim 20, comprising administering the one or more compounds of claim 1 to a human subject.
